(19) United States
(12) Patent Application Publication
(10) Pub. No.: US 2020/0291375 A1
(43) Pub. Date: Sep. 17, 2020

(54) FUNGAL PROTEASE MIXTURES AND USES THEREOF

(71) Applicant: BIO-CAT, INC., Troy, VA (US)

(72) Inventors: Kelly Tinker GREGORY, Troy, VA (US); Caroline BEST, Troy, VA (US); Christopher PENET, Troy, VA (US); Christopher SCHULER, Troy, VA (US)

(21) Appl. No.: 16/650,077

(22) PCT Filed: Sep. 24, 2018
(86) PCT No.: PCT/US2018/052484

§ 371 Date: Mar. 24, 2020

Related U.S. Application Data
(60) Provisional application No. 62/562,473, filed on Sep. 24, 2017.

(51) Int. Cl. C12N 9/62; C12Q 1/37; A23L 5/20; A23L 29/00

(52) U.S. Cl. CPC C12N 9/62; C12Q 1/37; C12Q 2521/537; A23L 29/06; C12Y 302/01001; A23L 5/25

(57) ABSTRACT

Fungal protease compositions, and more particularly, mixtures of Aspergillus proteases are provided. The disclosure also relates to protein hydrolysates, food and beverage products and dietary supplements produced using these Aspergillus protease mixtures, and methods of making and using the same.

### TECHNICAL FIELD

Novel fungal protease compositions, and more particularly, mixtures of Aspergillus proteases are provided. The disclosure also relates to protein hydrolysates, food and beverage products and dietary supplements produced using these Aspergillus protease mixtures, and methods of making and using the same.

### BACKGROUND

Raw protein sources may be hydrolyzed to produce peptides and/or free amino acids using naturally-occurring or recombinant proteolytic enzymes or by chemical decomposition. This hydrolysate may then be used for various purposes, e.g., as a seasoning, food additive or dietary supplement intended to promote nutrition, or as a precursor or component of another protein-related product. Proteases are generally characterized as exonucleases or endonucleases depending on whether they cleave peptide bonds of the terminal residues or internal residues of a polypeptide or oligopeptide. Proteases may also be labeled as specific to a particular residue (or residues) or nonspecific, based upon whether the proteolytic activity is dependent on a given signature being present in the sequence of the polypeptide or oligopeptide being digested.

Enzymatic digestion may proceed using a single proteolytic enzyme (e.g., a single, nonspecific exonuclease that may gradually digest a given polypeptide or oligopeptide). Alternatively, digestion may involve the use of a mixture of proteases that display different proteolytic activity profiles. Proteolytic enzyme cocktails known in the art include the Flavourzyme® enzyme mixture (a proteolytic enzyme preparation derived from A. oryzae which comprises at least five proteolytic components, each having an approximate molecular weight, respectively, selected from 23 kD, 27 kD, 31 kD, 32 kD, 35 kD, 38 kD, 42 kD, 47 kD, 53 kD, and 100 kD), as described in International Patent Application Publication No. WO1994/025580, and in Merz et al., “Flavourzyme, an enzyme preparation with industrial relevance: automated nine-step purification and partial characterization of eight enzymes.” Journal of agricultural and food chemistry 63.23 (2015): 5682-5693, the contents of each of which is incorporated herein by reference. The proper selection of an enzyme (or enzymes in a mixture) for production of a hydrolysate is important because the characteristics and properties of the hydrolysate will vary depending on the type and degree of proteolysis. For example, incomplete digestion may generate a hydrolysate enriched in oligopeptides or free amino acids which create a bitter taste or chalky mouthfeel, resulting in a product unusable for certain desirable purposes (e.g., an additive for food products). Other properties of proteolytic enzymes, such as stability, efficiency, cost, and compatibility with other common solvents/reagents are also highly relevant to the selection of a proteolytic enzyme or cocktail of enzymes for hydrolysate production. These limitations constrain the commercial or industrial use of particular enzymes and combinations thereof.

### SUMMARY OF VARIOUS EMBODIMENTS

In a general aspect, the present disclosure relates to combinations of proteases obtained from one or more members of the genus Aspergillus, such as A. oryzae. This combination of enzymes is capable of digesting protein from various sources to produce a hydrolysate enriched in essential amino acids and branched chain amino acids. In some exemplary aspects, the proteolytic enzyme mixtures described herein may be used to produce a hydrolysate that has improved flavor and/or mouthfeel compared to a hydrolysate prepared using currently available enzymes that often produce bitter and/or chalky hydrolysates. Moreover, the combination of enzymes described herein is stable and maintains activity over a broad range of temperatures and pH levels, providing additional options for commercial and industrial applications.

In other general aspects, the disclosure provides methods of preparing a protein hydrolysate from various protein sources using the disclosed enzyme mixtures, and in particular protein hydrolysates enriched in essential amino acids and/or branched chain amino acids.

In still other general aspects, food products, additives, dietary supplements and beverages comprising the protein hydrolysates described herein are provided.

Methods of using the disclosed protein hydrolysates are also provided, including methods of increasing exercise performance and/or decreasing muscle breakdown during exercise by administering a protein hydrolysate prepared described herein, alone or as part of a food product, dietary supplement or beverage.

Additional aspects will be readily apparent to one of skill in light of the totality of the disclosure.

### BRIEF DESCRIPTION OF THE DRAWINGS

FIG. 1A is a graph illustrating the relative activity of Sumizyme® FL-G across various pH levels.

FIG. 1B is a graph illustrating the residual activity of Sumizyme® FL-G across various pH levels.

FIG. 1C is a graph illustrating the relative activity of Sumizyme® FL-G across various temperature levels.

FIG. 1D is a graph illustrating the residual activity of Sumizyme® FL-G across various temperature levels.

FIG. 2A is a graph illustrating the relative activity of Sumizyme® LPL-G across various pH levels.

FIG. 2B is a graph illustrating the residual activity of Sumizyme® LPL-G across various pH levels.

FIG. 2C is a graph illustrating the relative activity of Sumizyme® LPL-G across various temperature levels.

FIG. 2D is a graph illustrating the residual activity of Sumizyme® LPL-G across various temperature levels.

FIG. 3 is a chart that illustrates a comparative analysis of the proteolytic activities of Sumizyme® FL-G, Sumizyme® LPL-G and OPTI-ZIOME™ Pro-ST, with an emphasis on differences in the levels of branched chain amino acids produced by each of these enzyme mixtures.

FIG. 4A is a graph illustrating the relative activity of OPTI-ZIOME™ Pro-ST across a range of pH levels.

FIG. 4B is a graph illustrating the effective of temperature on relative activity of OPTI-ZIOME™ Pro-ST.

FIG. 5A is a chart summarizing the amount of free amino acids present in a sample of pea protein digested by OPTI-ZIOME™ Pro-ST or Flavourzyme®.

FIG. 5B is a graph illustrating the amount of free amino acids present in a sample of pea protein digested by OPTI-ZIOME™ Pro-ST provided at 2, 5, 10 or 20 leucine aminopeptidase activity units (“LAPUs”) per gram of pea protein.

FIG. 5C is a graph illustrating the amount of free amino acids present in a sample of pea protein digested by 10 LAPUs/g of OPTI-ZIOME™ Pro-ST versus 10 LAPUs/g of Flavourzyme® under identical reaction conditions.

FIG. 6A is a chart summarizing the amount of free amino acids present in a sample of soy protein digested by OPTI-ZIOME™ Pro-ST or Flavourzyme®.

FIG. 6B is a graph illustrating the amount of free amino acids present in a sample of soy protein digested by OPTI-ZIOME™ Pro-ST provided at 2, 5, 10 or 20 LAPUs/g of soy protein.

FIG. 6C is a graph illustrating the amount of free amino acids present in a sample of soy protein digested by 10 LAPUs/g of OPTI-ZIOME™ Pro-ST versus 10 LAPUs/g of Flavourzyme® under identical reaction conditions.

FIG. 7A is a chart summarizing the amount of free amino acids present in a sample of whey protein digested by OPTI-ZIOME™ Pro-ST or Flavourzyme®.

FIG. 7B is a graph illustrating the amount of free amino acids present in a sample of whey protein digested by OPTI-ZIOME™ Pro-ST provided at 2, 5, 10 or 20 LAPUs/g of whey protein.

FIG. 7C is a graph illustrating the amount of free amino acids present in a sample of whey protein digested by 10 LAPUs/g of OPTI-ZIOME™ Pro-ST versus 10 LAPUs/g of Flavourzyme® under identical reaction conditions.

FIG. 8A is a chart summarizing the amount of free amino acids present in a sample of rice protein digested by OPTI-ZIOME™ Pro-ST or Flavourzyme®.

FIG. 8B is a graph illustrating the amount of free amino acids present in a sample of rice protein digested by OPTI-ZIOME™ Pro-ST provided at 2, 5, 10 or 20 LAPUs/g of rice protein.

FIG. 8C is a graph illustrating the amount of free amino acids present in a sample of rice protein digested by 10 LAPUs/g of OPTI-ZIOME™ Pro-ST versus 10 LAPUs/g of Flavourzyme® under identical reaction conditions.

FIG. 9A is a chart summarizing the amount of free amino acids present in a sample of hemp protein digested by OPTI-ZIOME™ Pro-ST or Flavourzyme®.

FIG. 9B is a graph illustrating the amount of free amino acids present in a sample of hemp protein digested by OPTI-ZIOME™ Pro-ST provided at 2, 5, 10 or 20 LAPUs/g of hemp protein.

FIG. 9C is a graph illustrating the amount of free amino acids present in a sample of hemp protein digested by 10 LAPUs/g of OPTI-ZIOME™ Pro-ST versus 10 LAPUs/g of Flavourzyme® under identical reaction conditions.

FIG. 10A is a graph illustrating the amount of free amino acids present in a sample of wheat protein digested by 5 LAPUs/g of OPTI-ZIOME™ Pro-ST versus 10 LAPUs/g of Flavourzyme® under identical reaction conditions.

FIG. 10B is a graph illustrating the amount of essential amino acids present in a sample of wheat protein digested by 5 LAPUs/g of OPTI-ZIOME™ Pro-ST versus 10 LAPUs/g of Flavourzyme® under identical reaction conditions.

FIG. 11A is a graph illustrating the amount of free amino acids present in a sample of casein protein digested by 5 LAPUs/g of OPTI-ZIOME™ Pro-ST versus 10 LAPUs/g of Flavourzyme® under identical reaction conditions.

FIG. 11B is a graph illustrating the amount of essential amino acids present in a sample of casein protein digested by 5 LAPUs/g of OPTI-ZIOME™ Pro-ST versus 10 LAPUs/g of Flavourzyme® under identical reaction conditions.

FIG. 12A is a chart summarizing the amount of essential amino acids present in a sample of pea protein digested by OPTI-ZIOME™ Pro-ST or Flavourzyme® provided at 2, 5, 10 or 20 LAPUs/g of pea protein.

FIG. 12B is a graph illustrating the amount of essential amino acids present in a sample of pea protein digested by OPTI-ZIOME™ Pro-ST provided at 2, 5, 10 or 20 LAPUs/g of pea protein.

FIG. 12C is a graph illustrating the amount of essential amino acids present in a sample of pea protein digested by 10 LAPUs/g of OPTI-ZIOME™ Pro-ST versus 10 LAPUs/g of Flavourzyme® under identical reaction conditions.

FIG. 13A is a chart summarizing the amount of essential amino acids present in a sample of soy protein digested by OPTI-ZIOME™ Pro-ST or Flavourzyme® provided at 2, 5, 10 or 20 LAPUs/g of soy protein.

FIG. 13B is a graph illustrating the amount of essential amino acids present in a sample of soy protein digested by OPTI-ZIOME™ Pro-ST provided at 2, 5, 10 or 20 LAPUs/g of soy protein.

FIG. 13C is a graph illustrating the amount of essential amino acids present in a sample of soy protein digested by 10 LAPUs/g of OPTI-ZIOME™ Pro-ST versus 10 LAPUs/g of Flavourzyme® under identical reaction conditions.

FIG. 14A is a chart summarizing the amount of essential amino acids present in a sample of whey protein digested by OPTI-ZIOME™ Pro-ST or Flavourzyme® provided at 2, 5, 10 or 20 LAPUs/g of whey protein.

FIG. 14B is a graph illustrating the amount of essential amino acids present in a sample of whey protein digested by OPTI-ZIOME™ Pro-ST provided at 2, 5, 10 or 20 LAPUs/g of whey protein.

FIG. 14C is a graph illustrating the amount of essential amino acids present in a sample of whey protein digested by 10 LAPUs/g of OPTI-ZIOME™ Pro-ST versus 10 LAPUs/g of Flavourzyme® under identical reaction conditions.

FIG. 15A is a chart summarizing the amount of essential amino acids present in a sample of rice protein digested by OPTI-ZIOME™ Pro-ST or Flavourzyme® provided at 2, 5 or 10 LAPUs/g of rice protein.

FIG. 15B is a graph illustrating the amount of essential amino acids present in a sample of rice protein digested by OPTI-ZIOME™ Pro-ST provided at 2, 5, 10 or 20 LAPUs/g of rice protein.

FIG. 15C is a graph illustrating the amount of essential amino acids present in a sample of rice protein digested by 10 LAPUs/g of OPTI-ZIOME™ Pro-ST versus 10 LAPUs/g of Flavourzyme® under identical reaction conditions.

FIG. 16A is a chart summarizing the amount of essential amino acids present in a sample of hemp protein digested by OPTI-ZIOME™ Pro-ST or Flavourzyme® provided at 2, 5, 10 or 20 LAPUs/g of hemp protein.

FIG. 16B is a graph illustrating the amount of essential amino acids present in a sample of hemp protein digested by OPTI-ZIOME™ Pro-ST provided at 2, 5, 10 or 20 LAPUs/g of hemp protein.

FIG. 16C is a graph illustrating the amount of essential amino acids present in a sample of hemp protein digested by 10 LAPUs/g of OPTI-ZIOME™ Pro-ST versus 10 LAPUs/g of Flavourzyme® under identical reaction conditions.

FIG. 17A is a chart summarizing the amount of branched chain amino acids present in a sample of pea protein digested by OPTI-ZIOME™ Pro-ST or Flavourzyme® provided at 2, 5, 10 or 20 LAPUs/g of pea protein.

FIG. 17B is a graph illustrating the amount of branched chain amino acids present in a sample of pea protein digested by OPTI-ZIOME™ Pro-ST provided at 2, 5, 10 or 20 LAPUs/g of pea protein.

FIG. 17C is a graph illustrating the amount of branched chain amino acids present in a sample of pea protein digested by 10 LAPUs/g of OPTI-ZIOME™ Pro-ST versus 10 LAPUs/g of Flavourzyme® under identical reaction conditions.

FIG. 18A is a chart summarizing the amount of branched chain amino acids present in a sample of soy protein digested by OPTI-ZIOME™ Pro-ST or Flavourzyme® provided at 2, 5, 10 or 20 LAPUs/g of soy protein.

FIG. 18B is a graph illustrating the amount of branched chain amino acids present in a sample of soy protein digested by OPTI-ZIOME™ Pro-ST provided at 2, 5, 10 or 20 LAPUs/g of soy protein.

FIG. 18C is a graph illustrating the amount of branched chain amino acids present in a sample of soy protein digested by 10 LAPUs/g of OPTI-ZIOME™ Pro-ST versus 10 LAPUs/g of Flavourzyme® under identical reaction conditions.

FIG. 19A is a chart summarizing the amount of branched chain amino acids present in a sample of whey protein digested by OPTI-ZIOME™ Pro-ST or Flavourzyme® provided at 2, 5, 10 or 20 LAPUs/g of whey protein.

FIG. 19B is a graph illustrating the amount of branched chain amino acids present in a sample of whey protein digested by OPTI-ZIOME™ Pro-ST provided at 2, 5, 10 or 20 LAPUs/g of whey protein.

FIG. 19C is a graph illustrating the amount of branched chain amino acids present in a sample of whey protein digested by 10 LAPUs/g of OPTI-ZIOME™ Pro-ST versus 10 LAPUs/g of Flavourzyme® under identical reaction conditions.

FIG. 20A is a chart summarizing the amount of branched chain amino acids present in a sample of rice protein digested by OPTI-ZIOME™ Pro-ST or Flavourzyme® provided at 2, 5, 10 or 20 LAPUs/g of rice protein.

FIG. 20B is a graph illustrating the amount of branched chain amino acids present in a sample of rice protein digested by OPTI-ZIOME™ Pro-ST provided at 2, 5, 10 or 20 LAPUs/g of rice protein.

FIG. 20C is a graph illustrating the amount of branched chain amino acids present in a sample of rice protein digested by 10 LAPUs/g of OPTI-ZIOME™ Pro-ST versus 10 LAPUs/g of Flavourzyme® under identical reaction conditions.

FIG. 21A is a chart summarizing the amount of branched chain amino acids present in a sample of hemp protein digested by OPTI-ZIOME™ Pro-ST or Flavourzyme® provided at 2, 5, 10 or 20 LAPUs/g of hemp protein.

FIG. 21B is a graph illustrating the amount of branched chain amino acids present in a sample of hemp protein digested by OPTI-ZIOME™ Pro-ST provided at 2, 5, 10 or 20 LAPUs/g of hemp protein.

FIG. 21C is a graph illustrating the amount of branched chain amino acids present in a sample of hemp protein digested by 10 LAPUs/g of OPTI-ZIOME™ Pro-ST versus 10 LAPUs/g of Flavourzyme® under identical reaction conditions.

FIG. 22A is a chart summarizing the amount of branched chain amino acids present in a sample of wheat protein digested by OPTI-ZIOME™ Pro-ST provided at 5 LAPUs/g of wheat protein or Flavourzyme® provided at 10 LAPUs/g of wheat protein.

FIG. 22B is a graph illustrating the amount of branched chain amino acids present in a sample of wheat protein digested by OPTI-ZIOME™ Pro-ST provided at 5 LAPUs/g of wheat protein or Flavourzyme® provided at 10 LAPUs/g of wheat protein.

FIG. 23A is a chart summarizing the amount of branched chain amino acids present in a sample of casein protein digested by OPTI-ZIOME™ Pro-ST provided at 5 LAPUs/g of wheat protein or Flavourzyme® provided at 10 LAPUs/g of casein protein.

FIG. 23B is a graph illustrating the amount of branched chain amino acids present in a sample of casein protein digested by OPTI-ZIOME™ Pro-ST provided at 5 LAPUs/g of wheat protein or Flavourzyme® provided at 10 LAPUs/g of casein protein.

FIG. 24A is a graph illustrating the results of a flavor preference test comparing assessors' preference for untreated protein versus protein treated with OPTI-ZIOME™ Pro-ST.

FIG. 24B is a graph illustrating the results of a flavor preference test comparing assessors' preference for protein treated with 2, 5 or 10 LAPUs/g of OPTI-ZIOME™ Pro-ST.

FIG. 25A is a graph illustrating the results of a solubility test comparing the solubility of untreated protein versus protein treated with OPTI-ZIOME™ Pro-ST.

FIG. 25B is a graph illustrating the results of a solubility test comparing the solubility of untreated protein, protein treated with 2 or 5 LAPUs/g of OPTI-ZIOME™ Pro-ST versus protein treated with 10 LAPUs/g of Flavourzyme®.

### DETAILED DESCRIPTION OF VARIOUS EMBODIMENTS

The present disclosure relates to proteolytic enzyme mixtures comprising a plurality of fungal proteases obtained from one or more members of the genus Aspergillus. The enzyme mixtures may be used to produce a protein hydrolysate enriched in essential amino acids and/or branched chain amino acids, and may also possess additional beneficial properties (e.g., less bitterness, improved flavor and/or mouthfeel) compared to protein hydrolysate produced using currently available proteases and protease mixtures. Additionally, methods of using these proteolytic enzyme mixtures, and various products (e.g., foods, beverages, dietary supplements) and other vehicles for administering the resulting protein hydrolysate are also provided. Methods of producing protein hydrolysates enriched with essential amino acids or branched chain amino acids from a single protein source (e.g., without the need for supplementation with additional essential amino acids or branched chain amino acids from another source) are also provided.

Proteins are high molecular weight polymers composed of multiple amino acid residues linked by peptide bonds. These bonds must be cleaved in order for protein to be absorbed and utilized by a human or other organism, with such cleavage typically being performed by endogenous proteolytic enzymes of the gastrointestinal tract that separate the polypeptides into its constituent free amino acids. Amino acids may be classified as essential or non-essential for any given organism, depending on whether an organism is capable of synthesizing the given amino acid. For a dietary regimen to be considered adequate for the support of normal physiological functions, it should contain all essential amino acids in the appropriate levels and in proper proportions. For humans, the nine essential amino acids are leucine, isoleucine, valine, methionine, tryptophan, phenylalanine, threonine, lysine and histidine.

Three of the essential amino acids (valine, leucine and isoleucine) have aliphatic side-chains with a branch, i.e., a central carbon atom bound to three or more carbon atoms. These branched chain amino acids (“BCAAs”) are particularly notable because these amino acids are an important nutritional factor for proper muscle physiology and metabolism. Reports further indicate that athletic and exercise performance may be improved by BCAA supplements. See e.g., Glynn et al., “Excess Leucine Intake Enhances Muscle Anabolic Signaling but Not Net Protein Anabolism in Young Men and Women.” The Journal of Nutrition. 2010. 140(11), 1970-1976; Sharp et al., “Amino Acid Supplements and Recovery from High-Intensity Resistance Training.” Journal of Strength and Conditioning Research. 2010. 24(4), 1125-1130. The remaining non-essential amino acids provide a source of metabolizable nitrogen required for the biosynthesis of proteins, purines, nucleic acids, and other metabolites.

In view of the above, there has been commercial interest in dietary supplements and food additives that contain essential amino acids and BCAAs (e.g., protein powders and energy drinks directed to athletes). These supplements are typically prepared by digesting a raw protein source (e.g., whey protein) using a proteolytic enzyme or combination of enzymes and then supplementing the end product with BCAAs or other essential amino acids obtained from a second process or source. The manufacturing of such products is therefore complicated by the fact that amino acids must typically be obtained from multiple sources and mixed together to obtain a product which has the desired profile and ratios (e.g., enriched in BCAAs).

The present disclosure provides proteolytic enzyme mixtures that simplify this process by generating protein hydrolysates already enriched in essential amino acids, and more particularly BCAAs. Use of these proteolytic enzyme mixtures reduces the complexity and manufacturing costs associated with having to obtain amino acids from different sources. Moreover, protein hydrolysates produced using these proteolytic enzyme mixtures have been found to have an improved taste, texture (e.g., mouthfeel) and solubility profiles compared to hydrolysates produced using known proteolytic enzymes and combinations thereof.

Protein hydrolysates produced using the present methods are therefore well-suited for use in commercial food products, dietary supplements, additives, and beverages. These food products and other vehicles may in turn be used by consumers, and athletes in particular, to provide nutrition, as well as athletic and/or exercise benefits.

### Proteolytic Enzyme Mixtures Comprising a Plurality of Aspergillus Proteases

In one general aspect, the present disclosure provides a proteolytic enzyme mixture comprising a plurality of fungal proteases obtained from one or more members of the genus Aspergillus. One or more of these enzymes may possess exonuclease, endonuclease and/or α-amylase activity, alone or operating in combination with one or both of the other enzymes. In some exemplary aspects, the mixture has proteolytic activity across a pH range spanning from 2.5 to 9.0, or any range of integer values therein. In some embodiments, the proteolytic activity of the mixture will be >60% across a pH range of 3.0 to 9.0, >80% across a pH range of 4.0 to 9.0, and/or >90% across a pH range of 5.0 to 9.0. The activity may also be >20% across a temperature range of 20 to 70° C., >60% across a temperature range of 40 to 70° C., >80% across a temperature range of 50 to 70° C., and/or >90% across a temperature range of 55 to 65° C. The proteolytic enzyme mixture may be capable of digesting a raw protein source (e.g., plant protein) and producing a protein hydrolysate enriched in essential amino acids and/or BCAAs when applied at a minimum of 1, 2, 5 10 or 20 LAPUs/g of protein. In some exemplary aspects, the proteolytic enzyme mixture is a three enzyme blend and may produce a hydrolysate with BCAA enrichment at a level several-fold larger than the BCAA level of hydrolysates prepared by digestion with only one or two of the three enzymes in the proteolytic enzyme mixture.

For example, in some embodiments, an enzyme mixture was prepared by combining Sumizyme® LPL-G (Fungal Protease from A. oryzae, CAS No.: 9025-49-4) and Sumizyme® FL-G (Protease/Peptidase from A. oryzae, CAS Nos.: 9001-61-0, 9074-07-1) at a ratio of 50:50 by weight, which were obtained from Shin Nihon Chemical Co. Ltd. The resulting mixture contains three enzymes, with approximate molecular weights of 25, 33 and 43 kDa, and displays exonuclease, endonuclease and α-amylase activity. As described herein, this combination has been shown to release essential amino acids, particularly BCAAs, in slightly acidic to neutral conditions (e.g., pH 3 to 9) while maintaining high activity. This combination of enzymes is referred to herein as “OPTI-ZIOME™ Pro-ST”.

OPTI-ZIOME™ Pro-ST was assayed for activity using a leucine aminopeptidase (“LAP”) assay, which is a commonly understand means for measuring protease activity. This assay is based on the enzymatic conversion of leucine p-nitroanilide (“LpNA”) to leucine and p-nitroaniline (“pNA”). The end product, pNA, is detected by a spectrophotometric reading at an absorbance of 405 nm. One LAP Unit (“LAPU”) is the amount of enzyme required to liberate 1 micromole of pNA per minute at 37° C. and pH 7. The LAP assay reveals that OPTI-ZIOME™ Pro-ST averages 350 LAPUs/g of protein being digested.

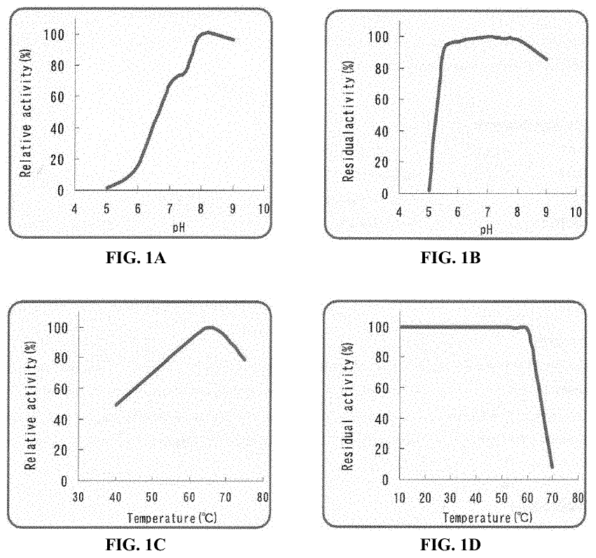

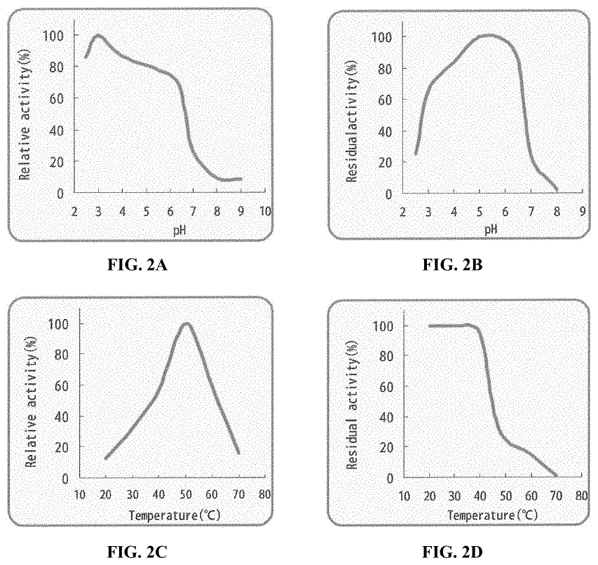

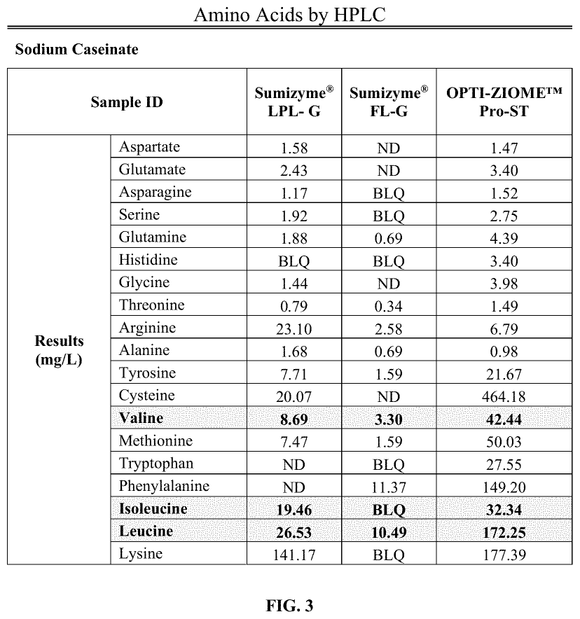

It is understood that a proteolytic enzyme mixture according to the disclosure may include the three A. oryzae enzymes identified above, which result from combining Sumizyme® LPL-G and Sumizyme® FL-G. It is further understood that the amounts or ratios of these three A. oryzae enzymes may be varied to produce a mixture having enhanced or reduced activity levels. For example, Sumizyme® LPL-G and Sumizyme® FL-G may be combined in a ratio of 25:75, 50:50, 75:50 or any other ratio which provides a desired activity level. FIGS. 1 and 2 provide graphs that illustrate the relative and residual activity levels of Sumizyme® FL-G (FIG. 1) and Sumizyme® LPL-G (FIG. 2) across various temperature and pH levels.

In some exemplary aspects, a proteolytic enzyme mixture according to the disclosure may include Aspergillopepsin-1, an aspartic endopeptidase produced by A. oryzae, e.g., the Aspergillopepsin-1 produced by A. oryzae strain ATCC 42149/RIB 40 represented by SEQ ID NO: 1 (UniProt Accession No. Q06902). The Aspergillopepsin-1 may have a polypeptide sequence identical to the amino acid sequence of SEQ ID NO: 1, or any fragment thereof (e.g., the fragment spanning position 78-404 of this sequence which is identified by UniProt as the mature form of this enzyme). In some exemplary aspects, a proteolytic enzyme mixture according to the disclosure may include an enzyme that shares at least 60, 65, 70, 75, 80, 85, 90, 95 or 98% sequence identity with the amino acid sequence of SEQ ID NO: 1, or with any fragment thereof, which retains the proteolytic activity of Aspergillopepsin-1.

In some exemplary aspects, a proteolytic enzyme mixture according to the disclosure may include an Aspergillopepsin-1 enzyme produced by an alternative strain of A. oryzae, e.g., the Aspergillopepsin-1 produced by the Yellow koji mold strain represented by SEQ ID NO: 2 (UniProt Accession No. P0CU33). In such exemplary aspects, the Aspergillopepsin-1 enzyme may be sequentially identical to the full-length A. oryzae strain enzyme, or an enzyme that shares at least 60, 65, 70, 75, 80, 85, 90, 95 or 98% sequence identity with the amino acid sequence of the full-length A. oryzae strain enzyme or a fragment thereof, which retains the proteolytic activity of Aspergillopepsin-1. For example, the Aspergillopepsin-1 enzyme may comprise a fragment which is identical to a fragment spanning positions 84-390 of the 390-residue full-length Aspergillopepsin-1 produced by the Yellow koji mold strain (SEQ ID NO: 2), or a variant which shares at least 60, 65, 70, 75, 80, 85, 90, 95 or 98% sequence identity with SEQ ID NO: 2.

As used herein, the term “sequence identity” refers to the degree to which two polynucleotide or amino acid sequences are identical (i.e., on a nucleotide-by-nucleotide or residue-by-residue basis, respectively) over the window of comparison. The percentage of sequence identity is calculated by comparing two optimally aligned sequences over the window of comparison, determining the number of positions at which the identical nucleic acid base (e.g., A, T, C, G for a polynucleotide sequence) occurs in both sequences to yield the number of matched positions, dividing the number of matched positions by the total number of positions in the window of comparison (i.e., the window size), and multiplying the result by 100 to yield the percentage of sequence identity. An equivalent calculation can be performed by comparing two aligned amino acid sequences.

FIG. 3 is a chart which illustrates a comparative analysis of the proteolytic activities of Sumizyme® FL-G, Sumizyme® LPL-G and OPTI-ZIOME™ Pro-ST, with an emphasis on differences in the levels of branched chain amino acids produced by each of these enzyme mixtures. Sodium caseinate was used as a protein substrate for this comparative assay. Three sodium caseinate samples were prepared as 5% solutions in water in stainless steel beakers, which were then adjusted to pH 6.7 and placed in a water bath set at 45° C. with overhead mixers placed in each of the solutions. Each of the three respective samples received either 1.5 LAPUs/g of OPTI-ZIOME™ Pro-ST, 3 LAPUs/g of Sumizyme® FL-G or 0.15 LAPUs/g of Sumizyme® LPL-G. These solutions were held at 45° C. for 1 hour with continuous mixing. After 1 hour, 5 mL of each sample was isolated and placed at 80° C. for 10 minutes to inactivate the enzymes. The remaining material was spray dried using a BUCHI B-290 Mini Spray Dryer.

The amino acid content of each sample was determined by high performance liquid chromatography (“HPLC”) using an Agilent 1100 HPLC with a gradient mobile phase beginning with 40 mM Na₂HPO₄ (pH 7.8), and changing to 45:45:10 acetonitrile:methanol:water running through a ZORBAX Eclipse-AAA 3.0×150 mm, 3.5 μm column with fluorescence detection. This method utilizes an online derivatization using o-phthalaldehyde for primary amino acids and 9-fluorenylmethyl chloroformate for secondary amino acids.

As illustrated by FIG. 3, the protein hydrolysate produced by the combination of three enzymes present in OPTI-ZIOME™ Pro-ST is surprisingly enriched in BCAAs compared to the protein hydrolysate produced by Sumizyme® FL-G or Sumizyme® LPL-G separately. For example, the amount of free leucine present in the OPTI-ZIOME™ Pro-ST hydrolysate is enriched by six-fold (172.25 mg/L) compared to the amount present in Sumizyme® LPL-G (26.53 mg/L) and by approximately seventeen-fold compared to the amount present in Sumizyme® FL-G (10.49 mg/L). The amounts of valine and isoleucine are similarly enriched, showing a substantial, non-additive increase in each case and evidence of synergistic effects that would not be expected from the amino acids levels produced by the individual components.

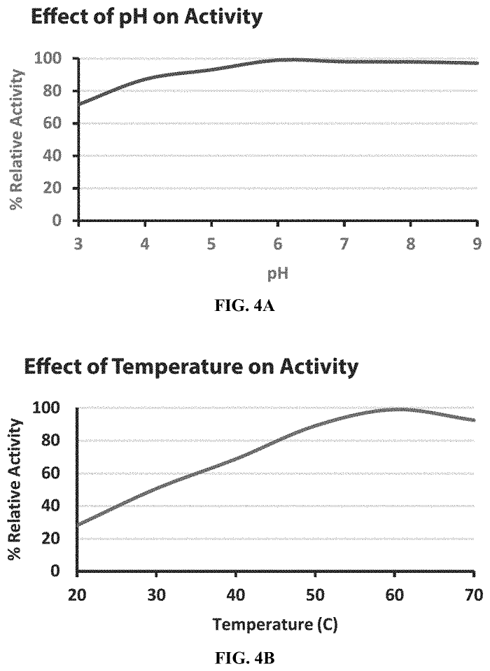

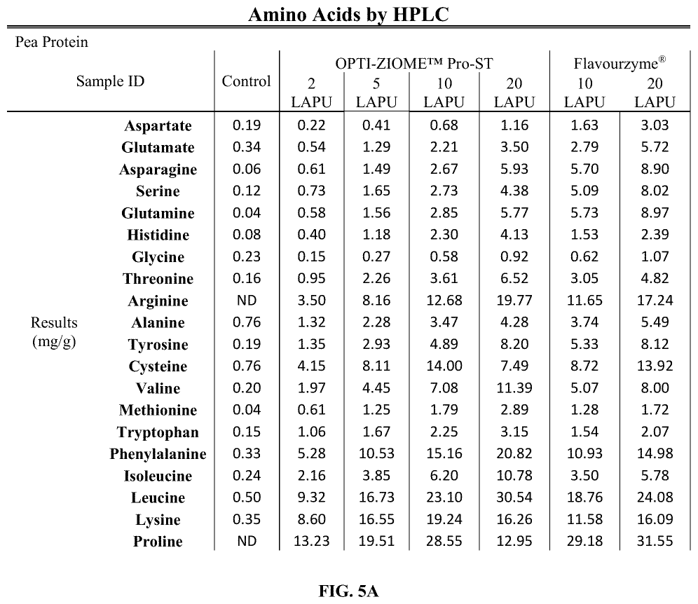

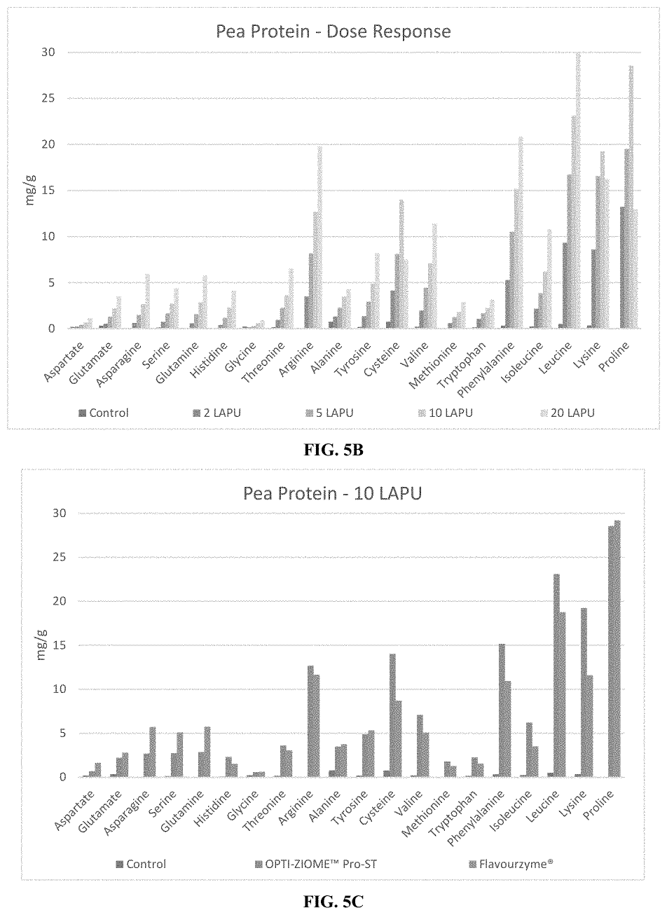

FIGS. 4 and 5 provide graphs illustrating the effect of pH and temperature on OPTI-ZIOME™ Pro-ST relative activity. As illustrated by these figures, OPTI-ZIOME™ Pro-ST maintains high levels of activity across a broad range of pH levels (e.g., pH 3-9) and temperatures (e.g., 20-70° C. with a peak at approximately 60° C.). It is understood that a user may perform protein digestion using OPTI-ZIOME™ Pro-ST at any temperature and/or pH level within these ranges, as is desirable for a given implementation. Temperature and/or pH levels outside of this range may also be suitable, with activity levels extrapolated from these graphs or measured according to routine methods known in the art.

In practice, OPTI-ZIOME™ Pro-ST will typically be used at 1-20 LAPUs/g of protein being digested. However, it is understood that this amount will be varied subject to routine optimization for a given application (e.g., additional LAPUs/g may be necessary at a lower incubation temperature for a given protein). Additional enzymes (e.g., proteases), coenzymes, cofactors, solvents, salts, etc., may be added to any of the protease enzyme mixtures disclosed herein as desired to improve or modify the digestion process as necessary or desired for a particular implementation.

In this exemplary aspect, OPTI-ZIOME™ Pro-ST is produced by combining Sumizyme® LPL-G and Sumizyme® FL-G, which results in a blend of three A. oryzae enzymes as described above. However, it is understood that homologous enzymes from other members of the Aspergillus genus may also be used. For example, the polypeptide sequence of the 25, 33 and 43 kDa enzymes present in this mixture may be determined by sequencing and used to identify putative homologs in other bacterial species (e.g., other members of the genus Aspergillus) having a sequence identify of greater than 80, 85, 90, 95, 96, 97, 98 or 99% compared to each of the respective polypeptide sequences and a combination featuring one or more of these putative homologs in place of the corresponding A. oryzae enzyme(s) to produce a proteolytic enzyme mixture in accordance with the disclosure. In some exemplary aspects, the proteolytic enzyme mixture may comprise a putative homolog as described above, which has exonuclease, endonuclease and/or α-amylase activity.

For example, a sequence search of the NCBI GenBank sequence database (www.ncbi.nlm.nih.gov/genbank/) using the BLASTP, PSI-BLAST or HMMER algorithms may identify one or more putative homologs in related Aspergillus species (e.g., A. niger) having >95% sequence identify compared to one of the three A. oryzae enzymes in OPTI-ZIOME™ Pro-ST. In some exemplary aspects, one or more of these putative homologs may be substituted in the mixture in place of the corresponding A. oryzae enzyme(s). It is understood that not all such combinations will be effective. However, the use of a high sequence identity cut-off, and optionally predicted domain architecture, may be used to identify homologs expected to share similar if not identical functionality. As a result, the search parameters above may be used to identify homologs and prepare additional protease enzyme mixtures in accordance with the disclosure following routine optimization. It is further understood that this approach is not limited to the genus Aspergillus, i.e., putative homologs from members of other genera may be identified and selected for use in the proteolytic enzyme mixtures described herein.

### Protein Hydrolysate Compositions and Methods of Preparation

Proteolytic enzyme mixtures described herein may be used to produce protein hydrolysates enriched in essential amino acids and/or BCAAs compared to protein hydrolysates produced by other protease enzymes and mixtures known in the art. Such hydrolysates may be produced from any raw protein source capable of digestion by a selected proteolytic enzyme mixture, including plant proteins (e.g., soy, hemp, rice, whey or pea protein), animal proteins (e.g., beef, chicken, or pork) and microbial proteins. Non-traditional protein sources such as insect protein (e.g., cricket protein) may also be used, as may proteins expressed from a recombinant organism (e.g., protein synthesized by a genetically-modified yeast culture).

As described above, proteolytic enzyme mixtures according to the disclosure may be used, in some embodiments, to produce hydrolysates having desirable properties such as enriched levels of essential amino acids or BCAAs. In some exemplary aspects, hydrolysates produced as described herein may have free leucine, isoleucine and/or valine levels which are several-fold higher than the levels of these free residues in protein hydrolyzed by any of the individual enzymes in the proteolytic enzyme mixture or by currently available proteases and mixtures. In some embodiments, such hydrolysates may be produced as a one-step process without supplementation from a secondary amino acid source (e.g., the initial hydrolysate may have a several-fold increase in one or more of these amino acids, avoiding the need for supplementation with additional BCAAs). In some exemplary aspects, hydrolysates produced as described herein may contain at least 10, 20, 30 or 40 mg/L of valine, at least 10, 20, 30 or 40 mg/L of isoleucine, and/or at least 10, 20, 30 or 40 mg/L of leucine. In some instances, the concentration of leucine in such hydrolysates may be further enriched to a level of at least 50, 100 or 150 mg/L.

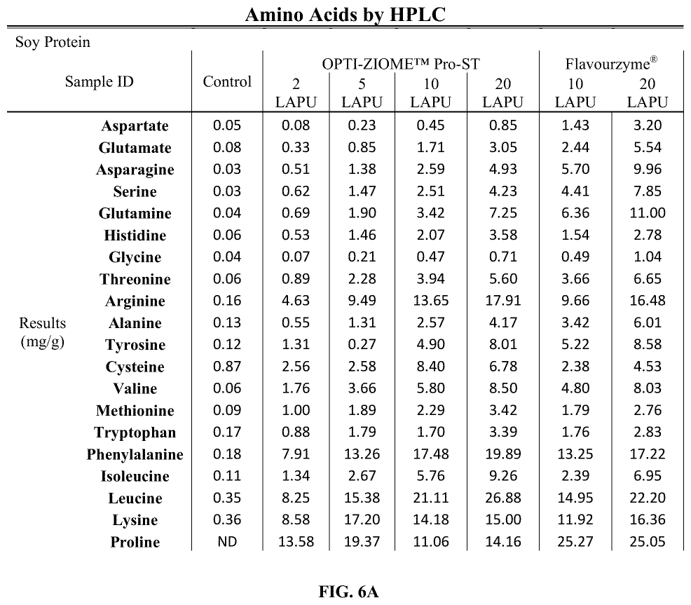

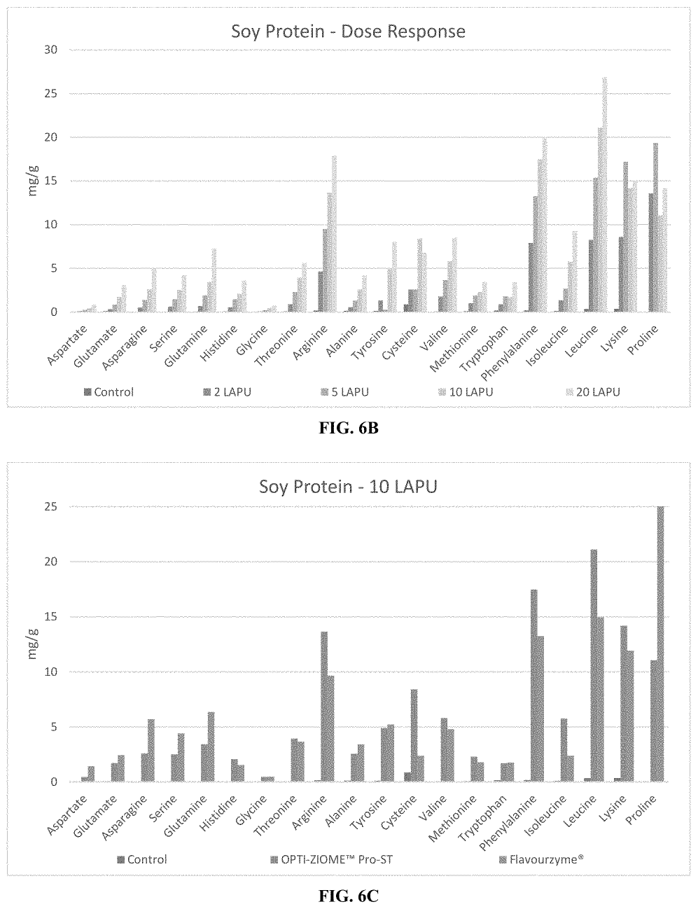

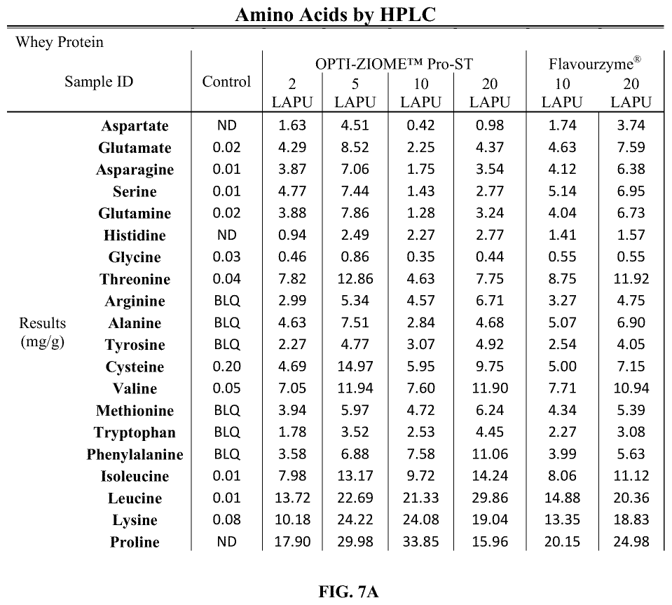

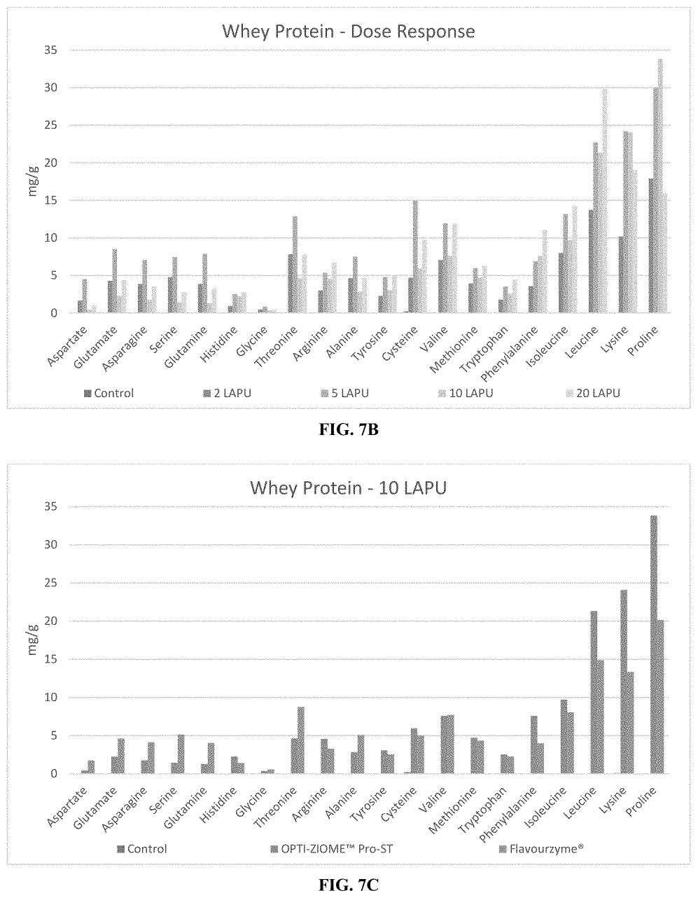

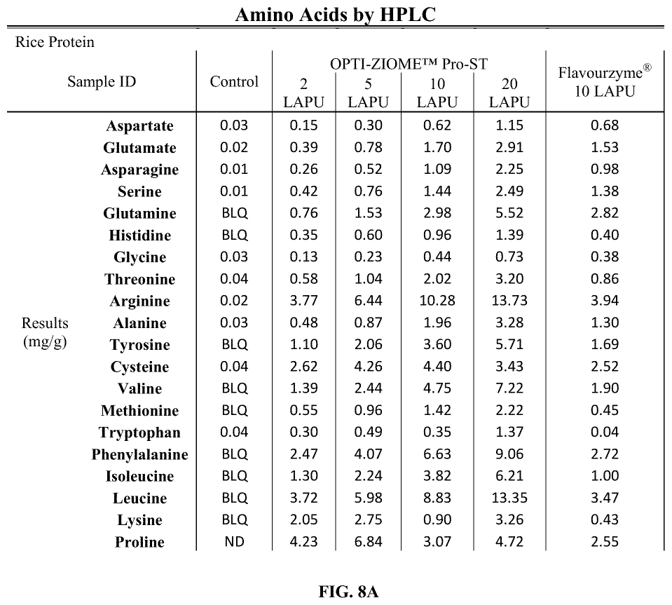

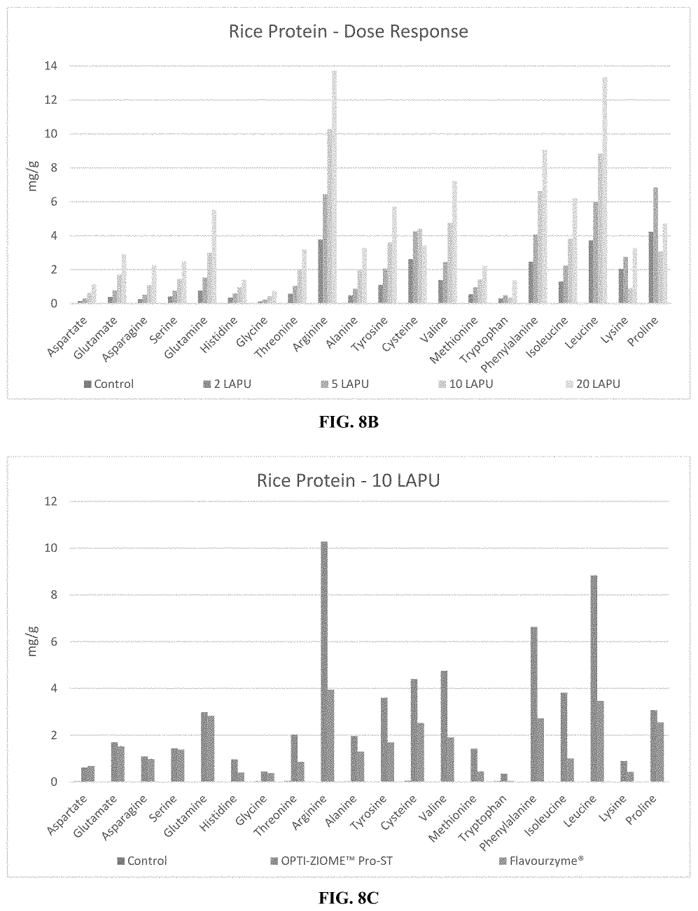

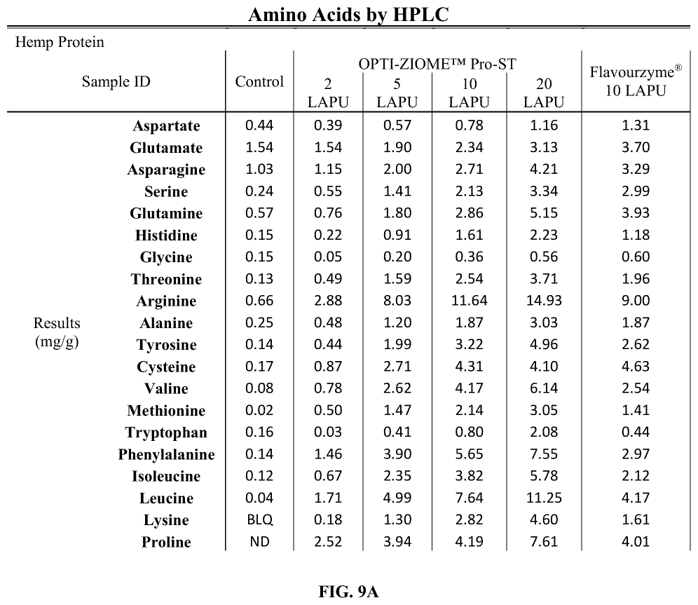

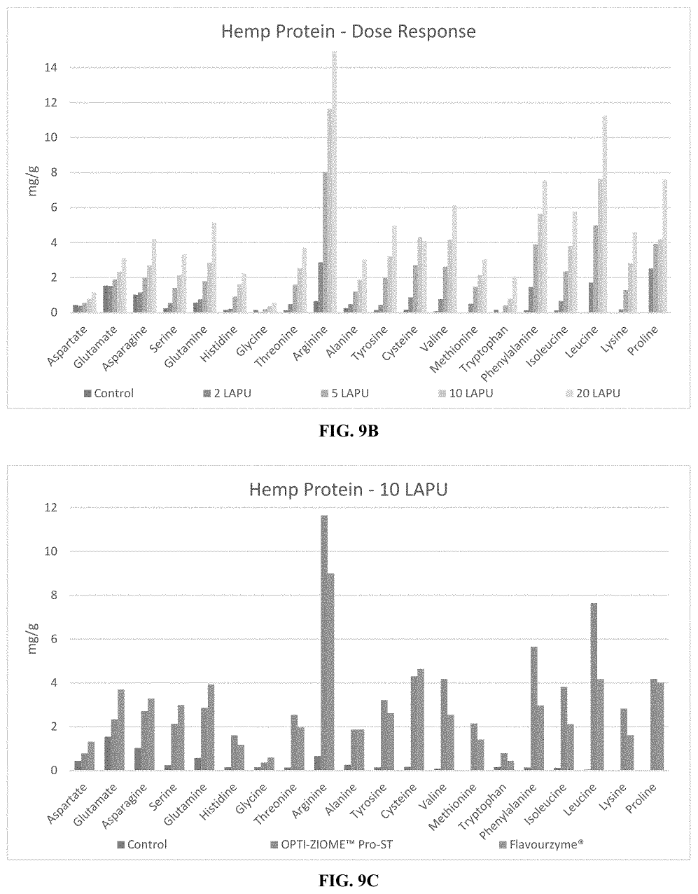

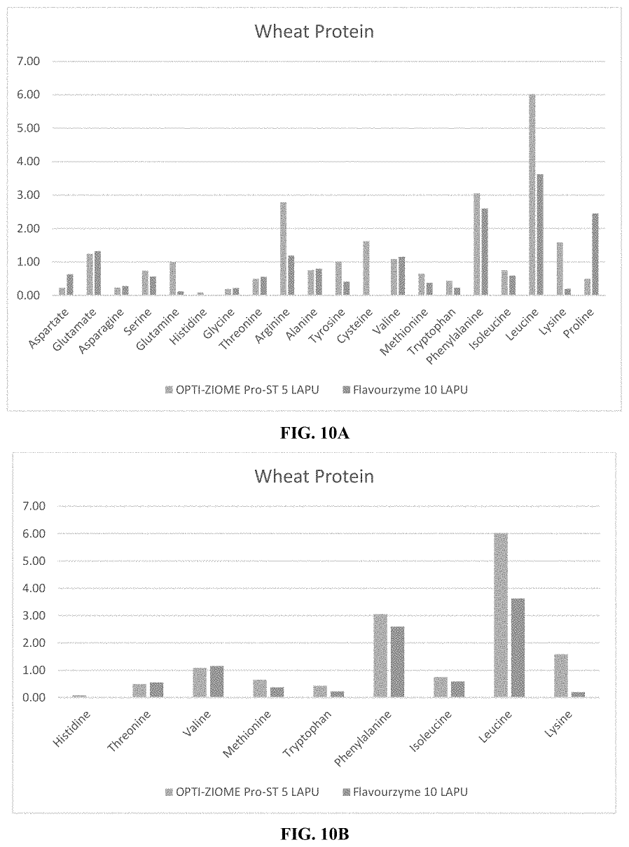

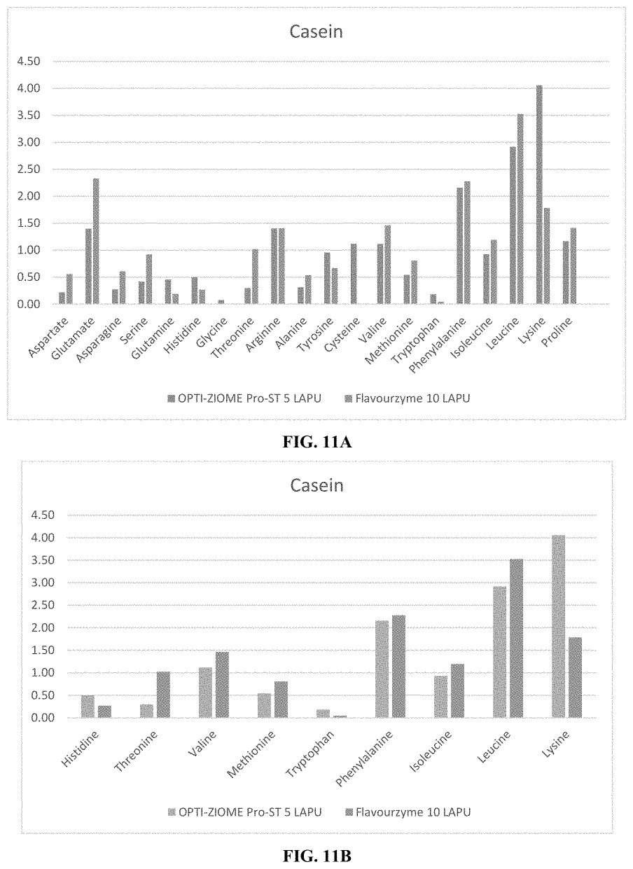

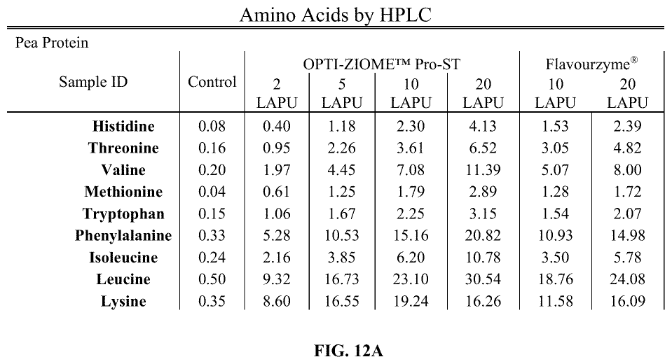

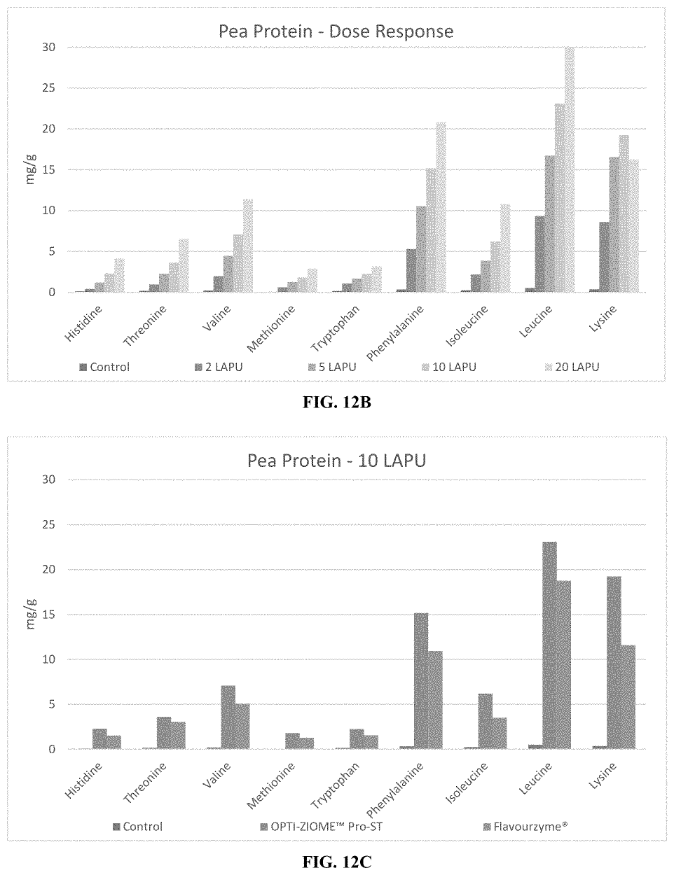

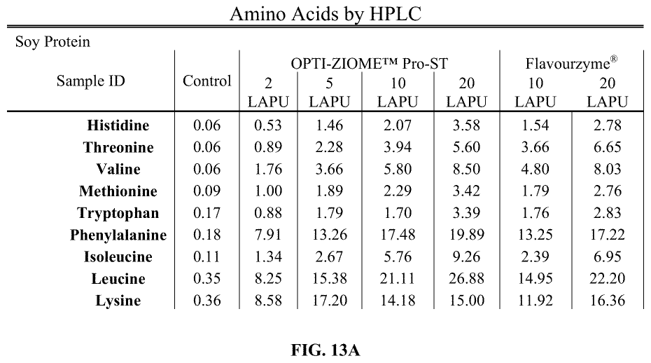

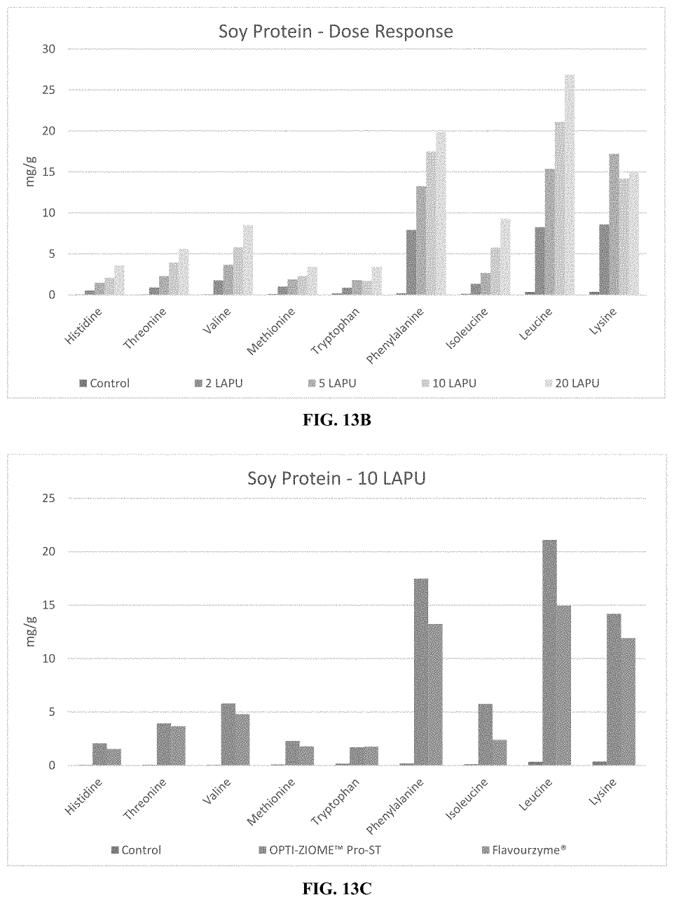

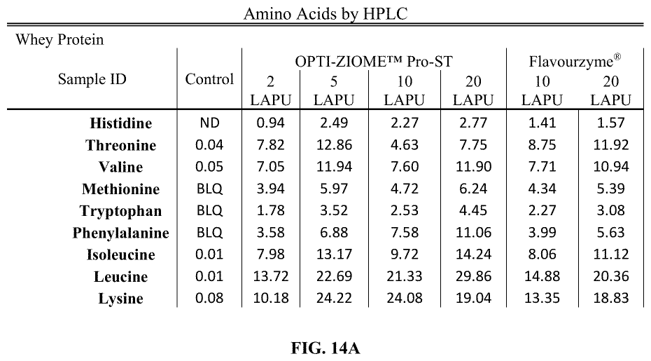

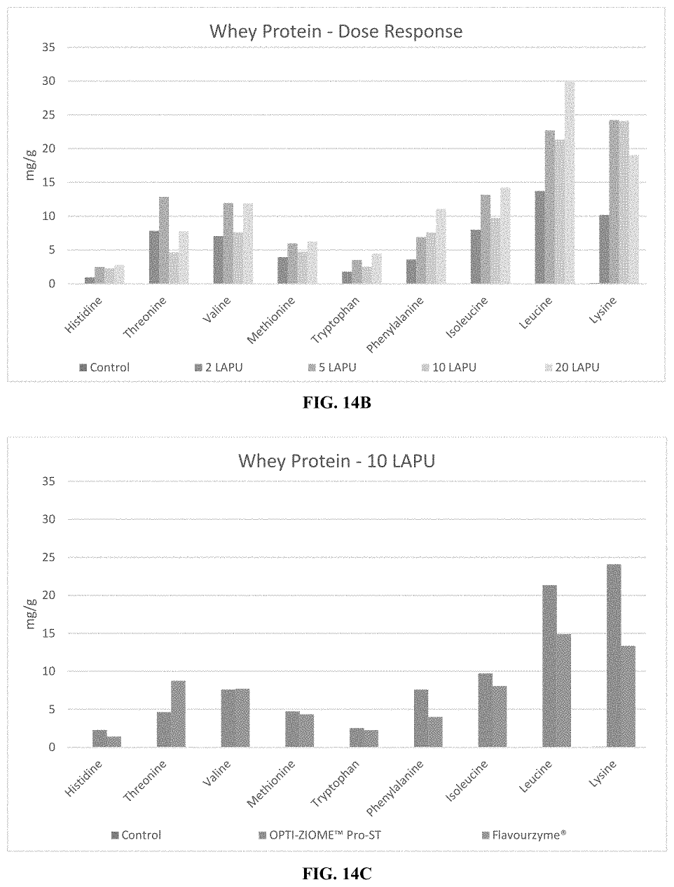

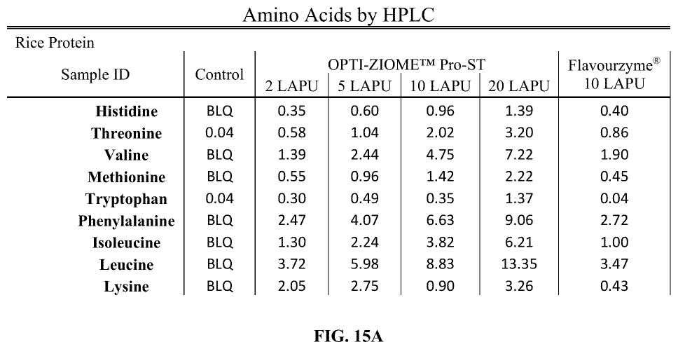

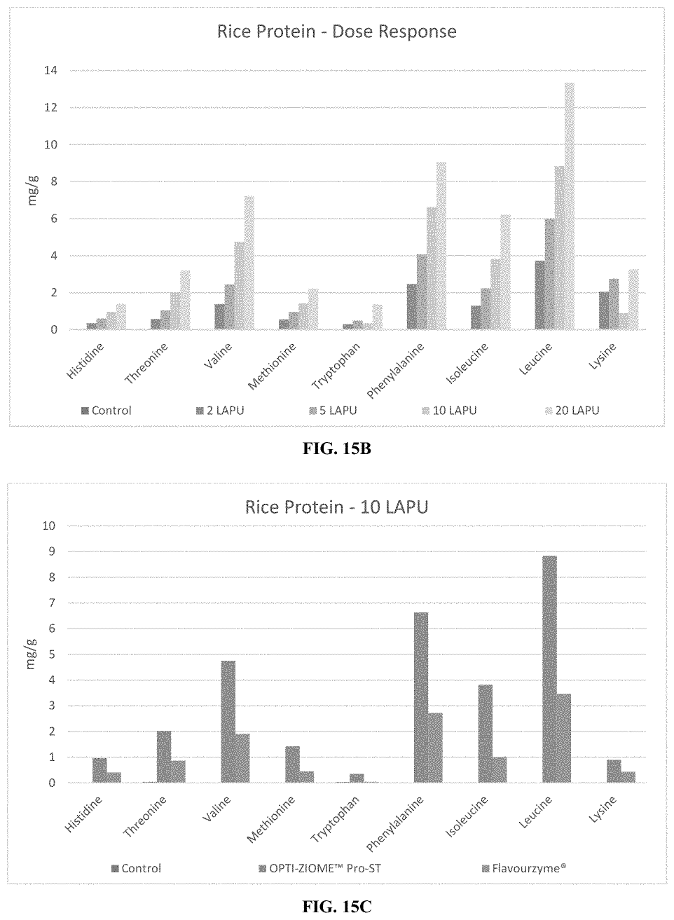

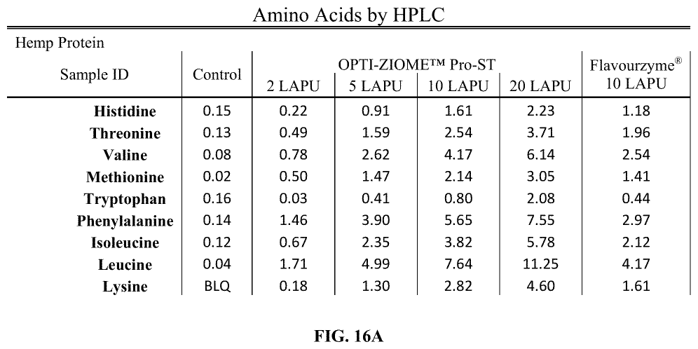

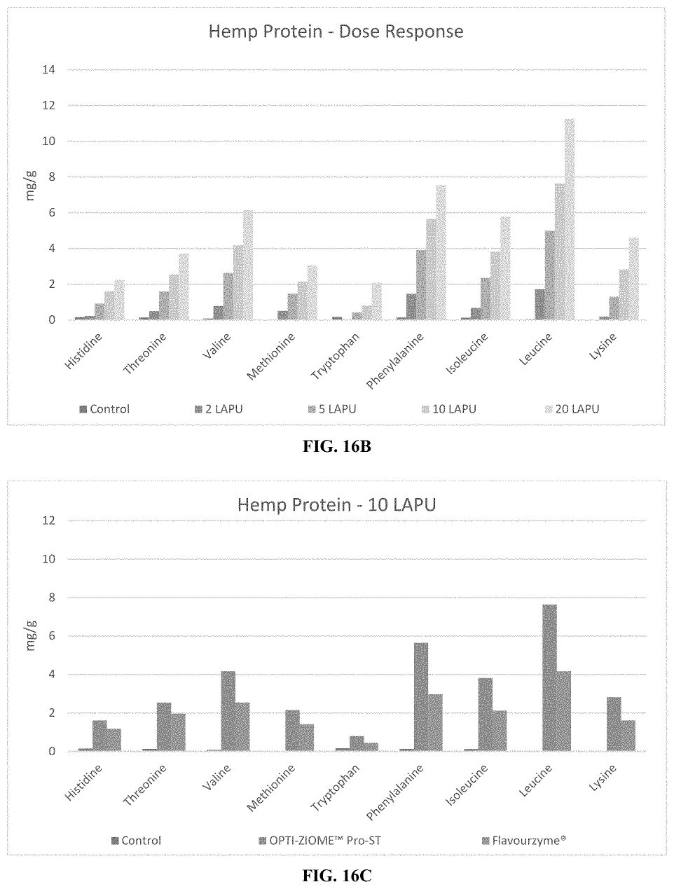

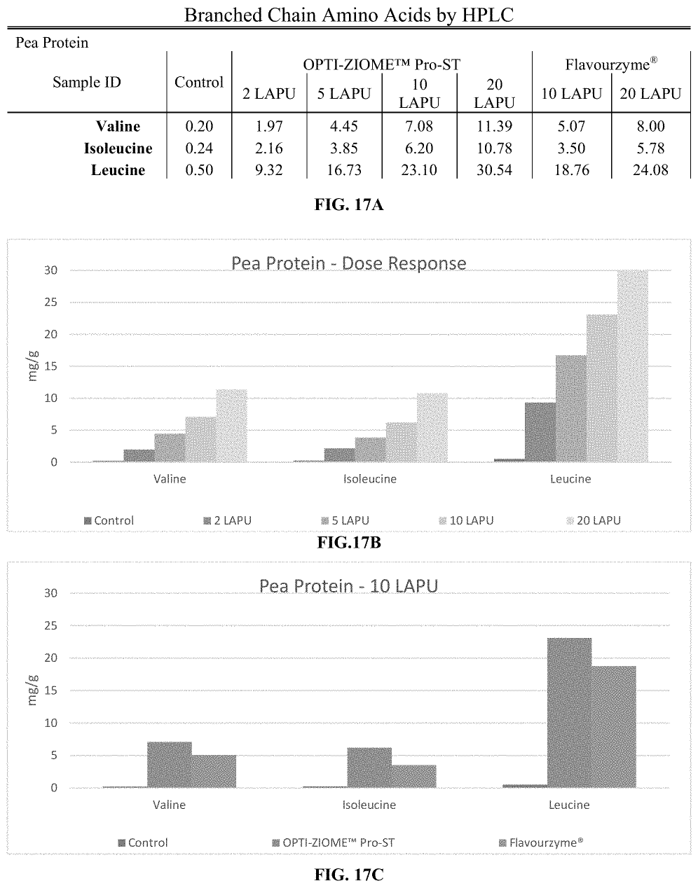

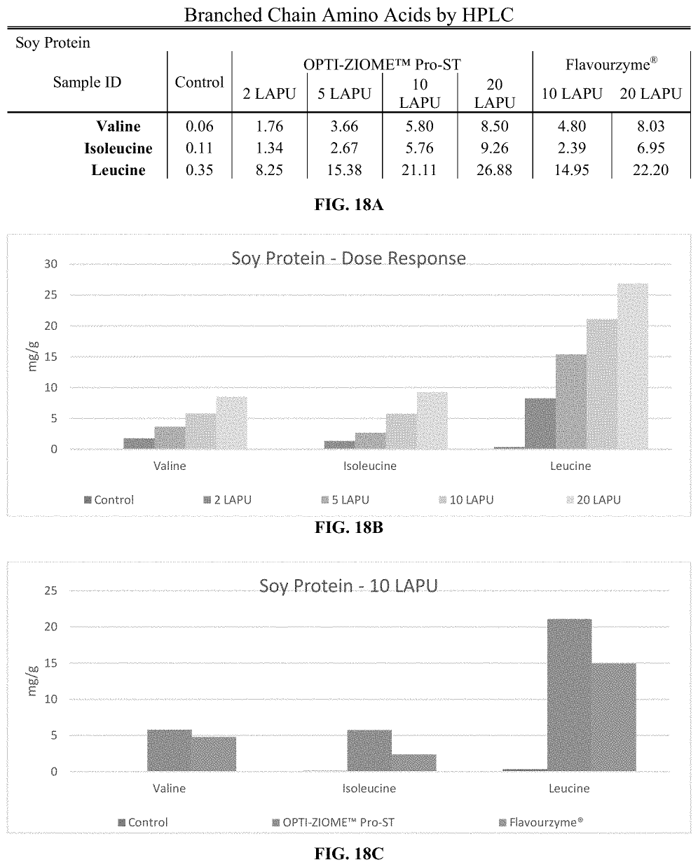

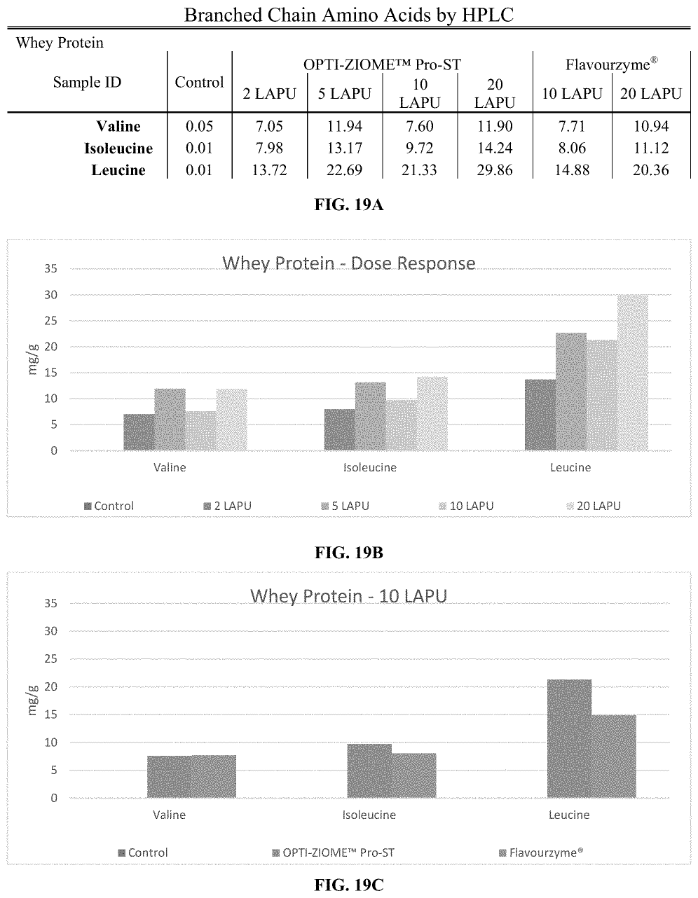

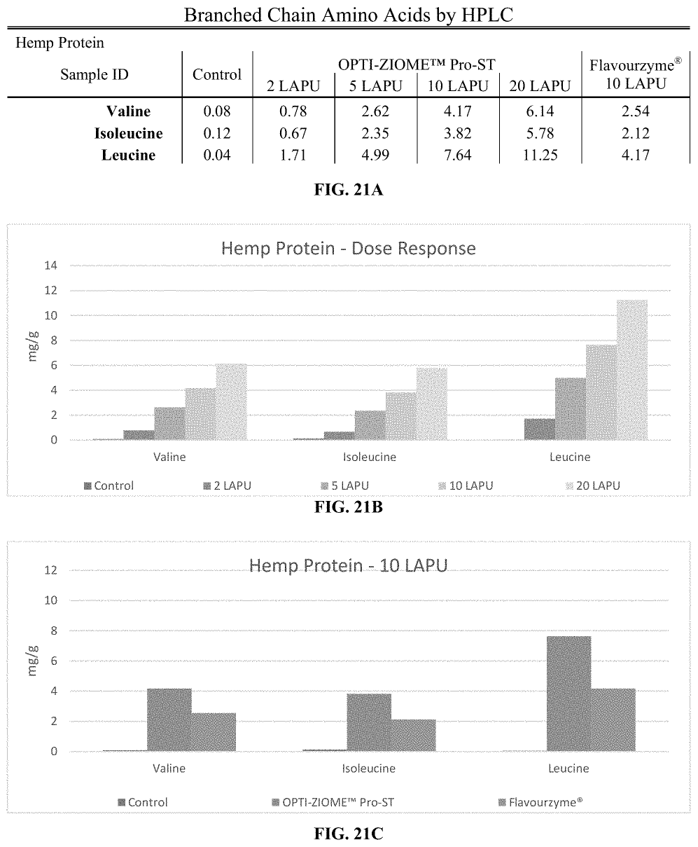

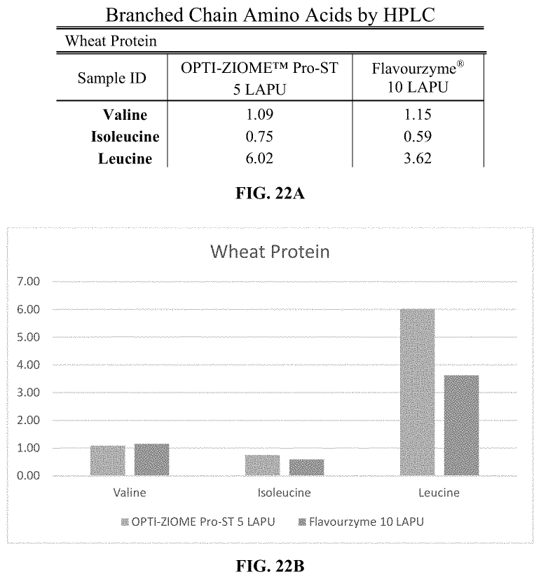

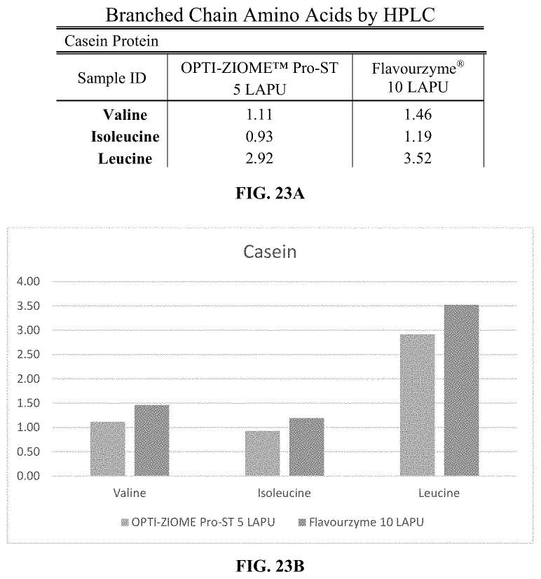

FIGS. 5-9 include charts and graphs summarizing the amount of free amino acids present in a sample of protein (pea, soy, whey, rice, or hemp protein) digested by OPTI-ZIOME™ Pro-ST or Flavourzyme® at 2, 5, or 10 LAPUs/g. For convenience, subsets of the charts shown by FIGS. 5A, 6A, 7A, 8A and 9A are excerpted as FIGS. 12C, 13C, 14C, 15C and 16C (highlighting the amount of essential amino acids) and FIGS. 17C, 18C, 19C, 20C and 21C (highlighting the amount of BCAAs). Similarly, FIGS. 10A and 11A include graphs summarizing the amount of free amino acids present in a sample of protein (wheat or casein protein) digested by 5 LAPUs/g of OPTI-ZIOME™ Pro-ST or 10 LAPUs/g Flavourzyme®. For convenience, subsets of the graphs shown by FIGS. 10A and 11A are excerpted as FIGS. 10B and 11B (highlighting the amount of essential amino acids) and FIGS. 22-23 (highlighting the amount of BCAAs).

All protein substrates used for these assays were prepared as 10% solutions in water in stainless steel beakers, which were then placed in a water bath set at 50° C. with overhead mixers placed in each of the solutions. No pH adjustment was performed; protein substrates were run at their native pH. The pH of the protein solutions was evaluated are as follows: Rice: 5.2, Hemp: 6.3, Soy: 6.6, Whey: 6.3, Pea: 6.6, Wheat: 3.9, and Casein: 4.9. OPTI-ZIOME™ Pro-ST was then added based on activity (LAPUs/g), with doses ranging from 2 to 20 LAPUs/g of protein substrate as indicated for each group in the respective charts and graphs. Flavourzyme® (Novozymes® protease from Aspergillus oryzae) was run under the same conditions at a dose of 10 LAPU/g of protein substrate on all five protein substrates and at a dose of 20 LAPU/g of protein substrate on three of the five protein substrates. The solutions were held at 50° C. for 2 hours with continuous mixing. After 2 hours, 5 mL of each sample was isolated and placed at 80° C. for 10 minutes to inactivate the enzymes. The remaining material was spray dried using a BUCHI B-290 Mini Spray Dryer.

The amino acid content of each sample was determined by HPLC using an Agilent 1100 HPLC with a gradient mobile phase beginning with 40 mM Na₂HPO₄ (pH 7.8) and changing to 45:45:10 acetonitrile:methanol:water running through a ZORBAX Eclipse-AAA 3.0×150 mm, 3.5 μm column with fluorescence detection. This method utilizes an online derivatization using o-phthalaldehyde for primary amino acids and 9-fluorenylmethyl chloroformate for secondary amino acids.

As illustrated by FIGS. 5-9 (pea, soy, whey, rice, and hemp protein), at the same dose (e.g., 10 LAPU/g) Flavourzyme® releases more non-essential amino acids (aspartate, glutamate, asparagine, serine, glutamine, and proline) than OPTI-ZIOME™ Pro-ST when digesting four of the five substrates. On all five of the substrates, OPTI-ZIOME™ Pro-ST releases more essential amino acids (histidine, threonine, valine, methinone, tryptophan, phenylalanine, isoleucine, leucine, and lysine) than Flavourzyme®. As a result, the three-enzyme combination present in OPTI-ZIOME™ Pro-ST is shown to consistently outperform the combination of at least five enzymes present in Flavourzyme®.

In this set of exemplary aspects, pea, soy, whey, rice, hemp, wheat and casein protein were assayed as protein sources for digestion. However, it is understood that other raw protein sources obtained from plants, fungi, bacteria or animals may also be digested using the protease enzyme mixtures disclosed herein. Similarly, the incubation time and temperature parameters described above may vary as necessary for a given application, while remaining in accordance with the present disclosure.

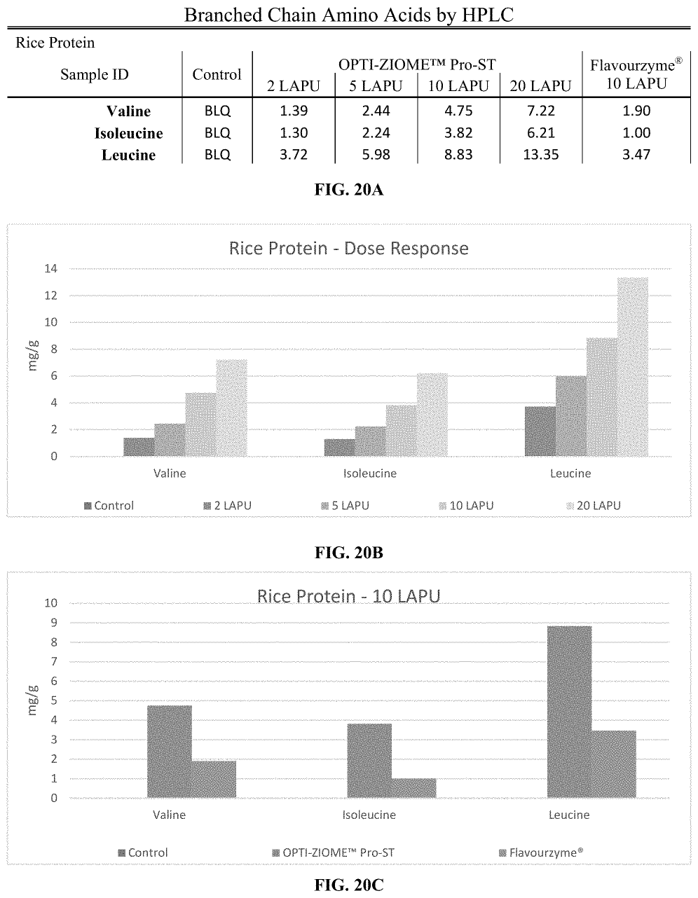

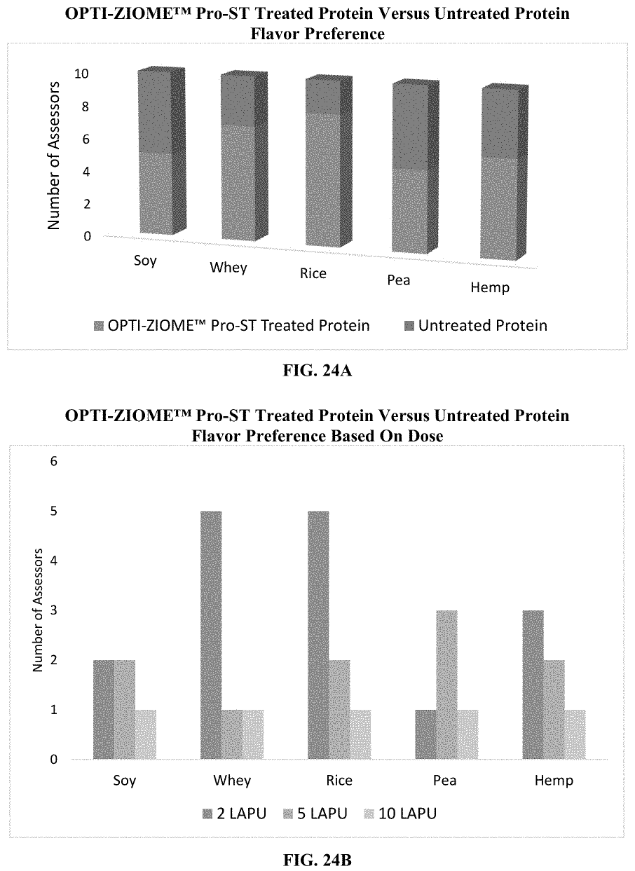

As illustrated by FIG. 24A, protein hydrolysates prepared using OPTI-ZIOME™ Pro-ST were preferred by a panel of assessors compared to untreated protein. The panel of assessors was screened by evaluating their ability to taste five basic flavors (sweet, salty, acidic, bitter, and neutral (water)) and also by tasting and ranking five bitter concentrations to discern variations in bitterness. Following this screening process, the screened assessors then compared the blind labeled hydrolysate samples to the raw (untreated) protein samples. The results were ranked, scored and then analyzed to determine the preference. In total 62% of the assessors in this study preferred the taste of OPTI-ZIOME™ Pro-ST over raw (untreated) protein. FIG. 20B provides dose-dependent flavor preference data, which indicates that assessors substantially preferred the taste of whey, rice and hemp protein treated with 2 LAPUs/g of OPTI-ZIOME™ Pro-ST. Soy protein treated with 2 or 5 LAPUs/g of OPTI-ZIOME™ Pro-ST was preferred equally and pea protein treated with 5 LAPUs/g of OPTI-ZIOME™ Pro-ST was preferred over samples treated with 2 or 10 LAPUs/g. These results confirm that OPTI-ZIOME™ Pro-ST at relatively low concentrations (e.g., 2 LAPUs/g) may be used to produce protein hydrolysate with desirable flavor profile from plant protein sources (e.g., whey or rice).

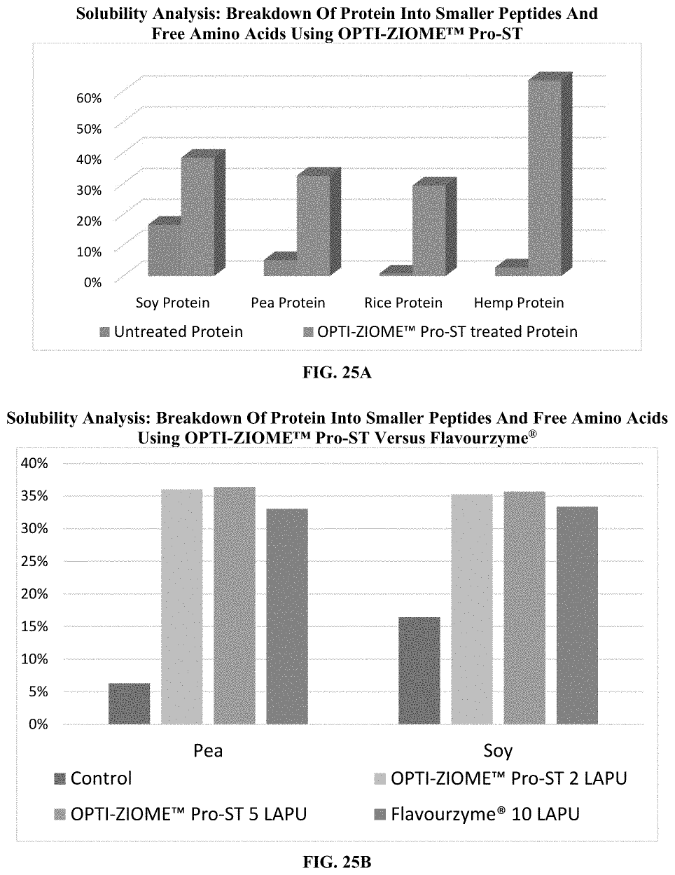

Solubility tests were also conducted on both the raw (untreated) proteins and hydrolysates of these proteins prepared using OPTI-ZIOME™ Pro-ST. Solubility was measured using the Lowry method for protein determination after dissolving the protein samples for one hour at pH 7 at ambient temperature. Percent solubility was calculated based on the amount of protein listed on the nutrition information label of each product. FIG. 25A summarizes the results of these solubility tests, which demonstrate that OPTI-ZIOME™ Pro-ST substantially increases the solubility of various raw proteins (e.g., soy, pea, rice or hemp protein), with a dramatic increase in solubility observed in the cases of hemp. FIG. 25B summarizes comparative solubility data for untreated pea and soy protein and samples treated with either 2 or 5 LAPUs/g of OPTI-ZIOME™ Pro-ST or 10 LAPUs/g of Flavourzyme®. As illustrated by these results, OPTI-ZIOME™ Pro-ST at 2 LAPUs/g provide superior solubility than Flavourzyme® at 10 LAPUs/g, providing further evidence of the improved performance of OPTI-ZIOME™ Pro-ST compared to commercially-available alternatives such as Flavourzyme®.

### Food Products, Ingredients or Additives, Dietary Supplements and Beverages Comprising Protein Hydrolysates

Protein hydrolysates produced according to the present disclosure may be used as food products, dietary supplements, as an ingredient or additive for a food product, in beverages, or in any other vehicle suitable for administration to or ingestion by a person or animal. In particular, hydrolysates according to the disclosure enriched in essential amino acids and/or BCAAs may be particularly desirable as food products, beverages or dietary supplements intended for athletes and subjects interested in improving exercise performance.

In some exemplary aspects, a protein hydrolysate may be prepared from a protein source (e.g., plant, animal or microbial-sourced raw protein) using any of the protease enzyme mixtures or methods of production described herein. The resulting hydrolysate may be optionally processed, such as by heat-inactivating the protease enzymes used to perform the digestion, chemically treating the mixture, and/or by filtering the mixture. The hydrolysate may also be optionally converted into a form more convenient for transport or storage (e.g., by drying, dehydrating or freeze-drying the hydrolysate). The hydrolysate may, subject to any such optional processing, be added to a food product, dietary supplement, beverage or any other vehicle suitable for administration to a human or animal, as indicated above. As indicated above, the relatively high solubility of protein hydrolysates produced using OPTI-ZIOME™ Pro-ST is particularly useful for beverages.

In some exemplary aspects, the hydrolysate is dried or dehydrated to form a protein powder enriched in essential amino acids and/or BCAAs. In other exemplary aspects, the hydrolysate is added to a food product such as a meal replacement or energy bar or beverage. The hydrolysate may be added to a vehicle as a powder or in liquid form, as is preferred for a given application.

### Methods of Using Protein Hydrolysates

Protein hydrolysates may be provided or administered to a human or animal in need of additional nutrition and/or to promote or provide a beneficial physiological effect. Protein hydrolysates enriched in essential amino acids and/or BCAAs are particularly useful as these classes of amino acid are associated with proper nutrition, muscle physiology and metabolism. As a result, protein hydrolysates produced according to the methods described herein may be used as a dietary supplement or as part of a treatment for humans or animals in order to improve nutrition or to improve athletic or exercise performance.

In some exemplary aspects, a protein hydrolysate as described herein may be administered to a subject in need thereof once, on a periodic basis or as part of any other regimen suitable to provide the subject with sufficient levels of one or more essential amino acids and/or BCAAs (e.g., to provide a desirable trait or reach a selected threshold associated with a desirable physiological state). The hydrolysate may be provided or administered as a food product, additive or ingredient to a food product, dietary supplement, beverage, or any other vehicle suitable which allows a subject to ingest or otherwise absorb amino acids in the hydrolysate.

Protein hydrolysates prepared using OPTI-ZIOME™ Pro-ST in accordance with any of the exemplary aspects above may be provided to a human or animal to promote nutrition or improved athletic or exercise performance, particularly hydrolysates enriched in BCAAs. It is understood that any such hydrolysates may be provided to a human or animal in need thereof as part of a food product, dietary supplement or beverage and may be provided in any amount necessary to provide a desirable function or outcome, with such amounts being the product of routine optimization depending on the nature of the individual or animal receiving the hydrolysate and/or the composition of the hydrolysate.

1. A proteolytic enzyme mixture comprising a plurality of fungal proteases from a member of the genus Aspergillus, wherein the plurality of fungal proteases comprises three enzymes having a molecular weight of approximately 25, 33 and 43 kDa, respectively, and the proteolytic enzyme mixture has exonuclease, endonuclease and α-amylase activity.

2. The proteolytic enzyme mixture of claim 1, wherein the three enzymes are from Aspergillus oryzae.

3. The proteolytic enzyme mixture of claim 1 or 2, wherein the mixture maintains proteolytic activity across a pH range of approximately 3.0 to 9.0.

4. The proteolytic enzyme mixture of any one of claims 1-3, wherein the mixture is stable over a temperature range of approximately 20 to 70° C.

5. The proteolytic enzyme mixture of any one of claims 1-4, in a dehydrated, powdered, granular or freeze dried form.

6. The proteolytic enzyme mixture of any one of claims 1-5, wherein the proteolytic enzyme mixture has an activity level of at least 100, 200 or 300 leucine aminopeptidase units (LAPUs) per gram of protein being digested, as measured by a leucine aminopeptidase assay.

7. The proteolytic enzyme mixture of any one of claims 1-6, wherein the proteolytic enzyme mixture has an activity level of at least 300 LAPUs per gram of protein being digested, as measured by a leucine aminopeptidase assay.

8. A method for preparing a protein hydrolysate, comprising:
contacting a composition comprising a protein with a proteolytic enzyme mixture comprising a plurality of fungal proteases from a member of the genus Aspergillus; and
digesting the protein using the proteolytic enzyme mixture, thereby obtaining the protein hydrolysate;
wherein the plurality of fungal proteases comprises three enzymes having a molecular weight of approximately 25, 33 and 43 kDa, and the proteolytic enzyme mixture has exonuclease, endonuclease and α-amylase activity.

9. The method of claim 8, wherein the three enzymes are obtained from Aspergillus oryzae.

10. The method of claim 8 or 9, wherein the digestion occurs at a pH within the range of approximately 3.0 to 9.0.

11. The method of any one of claims 8-10, wherein the digestion occurs at a temperature within the range of approximately 20 to 70° C.

12. The method of any one of claims 8-11, wherein the proteolytic enzyme mixture has an activity level of approximately 1-30 LAPUs per gram of protein being digested, as measured by a leucine aminopeptidase assay.

13. The method of any one of claims 8-12, wherein the proteolytic enzyme mixture has an activity level of approximately 1, 2, 5, 10, 15 or 20 LAPUs per gram of protein being digested, as measured by a leucine aminopeptidase assay.

14. The method of any one of claims 8-13, wherein the composition comprises protein obtained from a plant source.

15. The method of any one of claims 8-14, wherein the composition comprises protein obtained from one or more of the following sources: rice, hemp, soy, whey, peas, wheat, casein, gluten, corn gluten, or crickets.

16. The method of any one of claims 8-15, wherein the amount of essential amino acids in the protein hydrolysate is enriched compared to the amount obtained by contacting the composition with a proteolytic enzyme mixture comprising only one or two of the enzymes, under otherwise identical reaction conditions.

17. The method of any one of claims 8-16, wherein the amount of branched chain amino acids in the protein hydrolysate is enriched compared to the amount obtained by contacting the composition with a proteolytic enzyme mixture comprising only one or two of the enzymes, under otherwise identical reaction conditions.

18. The method of any one of claims 8-17, wherein the amount of essential amino acids in the protein hydrolysate is enriched compared to the amount obtained by contacting the composition with Flavourzyme®, when the Flavourzyme® is present in an amount which provides a LAPU activity level equivalent to the proteolytic enzyme mixture as measured by a leucine aminopeptidase assay, under otherwise identical reaction conditions.

19. The method of any one of claims 8-18, wherein the amount of branched chain amino acids in the protein hydrolysate is enriched compared to the amount obtained by contacting the composition with Flavourzyme®, when the Flavourzyme® is present in an amount which provides a LAPU activity level equivalent to the proteolytic enzyme mixture as measured by a leucine aminopeptidase assay, under otherwise identical reaction conditions.

20. The method of any one of claims 8-19, wherein the protein hydrolysate has increased solubility compared to a protein hydrolysate obtained by contacting the composition with Flavourzyme®, when the Flavourzyme® is present in an amount which provides a LAPU activity level equivalent to the proteolytic enzyme mixture as measured by a leucine aminopeptidase assay, under otherwise identical reaction conditions.

21. The method of any one of claims 8-20, wherein the protein hydrolysate is less bitter than:
(a) a protein hydrolysate obtained by contacting the composition with Flavourzyme®, when the Flavourzyme® is present in an amount which provides an activity level equivalent to the proteolytic enzyme mixture as measured by a leucine aminopeptidase assay, under otherwise identical reaction conditions; and/or
(b) the protein, untreated by the proteolytic enzyme mixture.

22. The method of any one of claims 8-21, wherein the three enzymes are present in the proteolytic enzyme mixture in a synergistically effective amount.

23. A protein hydrolysate produced by digesting a composition comprising a protein using a proteolytic enzyme mixture comprising a plurality of fungal proteases obtained from a member of the genus Aspergillus, wherein the plurality of fungal proteases comprises three enzymes having a molecular weight of approximately 25, 33 and 43 kDa, and the proteolytic enzyme mixture has exonuclease, endonuclease and α-amylase activity.

24. The protein hydrolysate of claim 23, wherein the three enzymes are obtained from Aspergillus oryzae.

25. The protein hydrolysate of claim 23 or 24, in a dehydrated, powdered, granular or freeze dried form.

26. The protein hydrolysate of any one of claims 23-25, wherein the composition comprises protein obtained from a plant source.

27. The protein hydrolysate of any one of claims 23-26, wherein the composition comprises protein obtained from one or more of the following: rice, hemp, soy, whey, peas, wheat, casein, gluten, corn gluten, or crickets.

28. A food product, dietary supplement or beverage comprising the protein hydrolysate of any one of claims 23-27.

29. A dietary supplement comprising the protein hydrolysate of any one of claims 23-27 in a dehydrated, powdered, granular or freeze dried form.

30. A method of increasing exercise performance and/or decreasing muscle breakdown during exercise, comprising:
administering the protein hydrolysate of any one of claims 23-27 to a subject in need thereof in an amount sufficient to increase exercise performance and/or decrease muscle breakdown during exercise.

31. The method of claim 30, wherein the protein hydrolysate is provided as a food product, food ingredient, food additive, dietary supplement or a beverage.

32. The proteolytic enzyme mixture of any one of claims 1-7, wherein the plurality of fungal proteases comprises an enzyme having a polypeptide sequence identical to either of SEQ ID NOs: 1 or 2, or a fragment thereof.

33. The proteolytic enzyme mixture of any one of claims 1-7, wherein the plurality of fungal proteases comprises an enzyme having a polypeptide sequence which shares at least 60, 65, 70, 75, 80, 85, 90, 95, or 98% sequence identity with either of SEQ ID NOs: 1 or 2, or a fragment thereof.

34. The method of any one of claims 8-22, wherein the plurality of fungal proteases comprises an enzyme having a polypeptide sequence identical to either of SEQ ID NOs: 1 or 2, or a fragment thereof.

35. The method of any one of claims 8-22, wherein the plurality of fungal proteases comprises an enzyme having a polypeptide sequence which shares at least 60, 65, 70, 75, 80, 85, 90, 95, or 98% sequence identity with either of SEQ ID NOs: 1 or 2, or a fragment thereof.

36. The protein hydrolysate of any one of claims 23-27, wherein the plurality of fungal proteases comprises an enzyme having a polypeptide sequence identical to either of SEQ ID NOs: 1 or 2, or a fragment thereof.

37. The protein hydrolysate of any one of claims 23-27, wherein the plurality of fungal proteases comprises an enzyme having a polypeptide sequence which shares at least 60, 65, 70, 75, 80, 85, 90, 95, or 98% sequence identity with either of SEQ ID NOs: 1 or 2, or a fragment thereof.

38. The food product, dietary supplement, or method of any one of claims 28-31, wherein:
a) the plurality of fungal proteases comprises an enzyme having a polypeptide sequence identical to either of SEQ ID NOs: 1 or 2, or a fragment thereof; and/or
b) the plurality of fungal proteases comprises an enzyme having a polypeptide sequence which shares at least 60, 65, 70, 75, 80, 85, 90, 95, or 98% sequence identity with either of SEQ ID NOs: 1 or 2, or a fragment thereof.


SEQUENCE LISTING (2 sequences)

SEQ ID NO: 1 - protein, 404 aa, Aspergillus oryzae (strain ATCC 42149 / RIB 40
MVILSKVAAVAVGLSTVASALPTGPSHSPHARRGFTINQITRQTARVGPKTASFPAIYSRALAKYGGTVPAHLKSAVASGHGTVVTSPEPNDIEYLTPVNIGGTTLNLDFDTGSADLWVFSEELPKSEQTGHDVYKPSGNASKIAGASWDISYGDGSSASGDVYQDTVTVGGVTAQGQAVEAASKISDQFVQDKNNDGLLGLAFSSINTVKPKPQTTFFDTVKDQLDAPLFAVTLKYHAPGSYDFGFIDKSKFTGELAYADVDDSQGFWQFTADGYSVGKGDAQKAPITGIADTGTTLVMLDDEIVDAYYKQVQGAKNDASAGGYVFPCETELPEFTVVIGSYNAVIPGKHINYAPLQEGSSTCVGGIQSNSGLGLSILGDVFLKSQYVVFDSQGPRLGFAAQA

SEQ ID NO: 2 - protein, 390 aa, Aspergillus oryzae (Yellow koji mold)
MVNTSLLAALTAYAVAVSAAPTAPQVKGFSVNQVAVPKGVYRHPAAQLAKAYGKYHATVPTQVAAAAAATGSVTTNPTSNDEEYITQVTVGDDTLGLDFDTGSADLWVFSSQTPSSERSGHDYYTPGSSAQKIDGATWSISYGDGSSASGDVYKDKVTVGGVSYDSQAVESAEKVSSEFTQDTANDGLLGLAFSSINTVQPTPQKTFFDNVKSSLSEPIFAVALKHNAPGVYDFGYTDSSKYTGSITYTDVDNSQGFWGFTADGYSIGSDSSSDSITGIADTGTTLLLLDDSIVDAYYEQVNGASYDSSQGGYVFPSSASLPDFSVTIGDYTATVPGEYISFADVGNGQTFGGIQSNSGIGFSIFGDVFLKSQYVVFDASGPRLGFAAQA